(19) United States
(12) Patent Application Publication
(10) Pub. No.: US 2005/0064465 A1
(43) Pub. Date: Mar. 24, 2005

(54) Continuous and non-continuous flow bioreactor

(75) Inventors: Roger Dettloff, Emerald Hills, CA (US); Celeste Kirby, San Jose, CA (US); Erik Gentalen, Redwood City, CA (US); Monica Rosoff, Half Moon Bay, CA (US)

Correspondence Address: CALIPER LIFE SCIENCES, INC., 605 FAIRCHILD DRIVE, MOUNTAIN VIEW, CA, 94043-2234, US

(73) Assignee: Caliper Life Sciences, Inc., Mountain View, CA (US)

(21) Appl. No.: 10/884,170

(22) Filed: Jul. 2, 2004

Related U.S. Application Data
(60) Provisional application No. 60/484,729, filed on Jul. 2, 2003.

(51) Int. Cl. C12Q 1/68; C12P 19/34

(52) U.S. Cl. 435/6; 435/91.2

(57) ABSTRACT

Methods and systems for performing continuous amplification of RNA and other nucleic acids are provided. Expression profiling using the continuous flow RNA amplification systems are also provided.

### STATEMENT REGARDING GOVERNMENT SUPPORT

This application was funded in part by NIH grant R44 GM062033-02. The government has certain rights in this application.

### BACKGROUND OF THE INVENTION

RNA production is central to all of biology. As has been understood for roughly half a century, messenger RNA (mRNA) is translated in the cell into proteins, which carry out most cellular operations. For example, in eukaryotes, mRNA is typically produced from nuclear RNA (nRNA), which is an RNA copy of a region of genomic DNA, by various splicing mechanisms. RNAs in general are typically encoded by genomic DNAs (gDNAs), with either mRNA or nRNA being produced by transcription of such DNA. This paradigm of DNA to RNA to protein is sometimes referred to as the “central paradigm” of molecular biology. Despite a few variations, such as those practiced by various RNA viruses (which can, e.g., have an RNA genome that is reverse transcribed into DNA and then replicated by transcription of the DNA back into RNA), this paradigm describes a basic way in which organisms encode cellular functions. See also, Alberts et al. (2002) Molecular Biology of the Cell, 4^{th} Edition Taylor and Francis, Inc., ISBN: 0815332181 (“Alberts”), and Lodish et al. (1999) Molecular Cell Biology, 4^{th} Edition W H Freeman & Co, ISBN: 071673706X (“Lodish”).

Detection of RNA types and levels of expression provide a basic tool for molecular biology and molecular medicine. For example, somatic or germline polymorphisms and/or mutations can be identified by detecting the polymorphism/mutation in RNA derived from a relevant individual (e.g., from a tissue or cell of the individual). Similarly, the level of RNA expression in a cell or tissue can be diagnostic of disease, or, e.g., of the cell or tissue type that the RNA is expressed in. See also, Alberts and Lodish, id.

Because there is a direct correlation between RNA expression and cellular and organismal function, a number of methods have been developed for detecting RNAs of interest. These methods all face various difficulties, derived, in part, from problems surrounding RNA manipulations generally. For example, enzymes that degrade RNAs are ubiquitous in the environment, causing degradation of RNA samples. Similarly, chemicals used to inhibit RNAse enzymes actually modify the RNA, making it unsuitable for certain further processing steps (e.g., reverse transcription and cloning).

Thus, rather than simply performing a northern blot for direct MRNA detection of mRNA in cells, various reverse transcription/amplification methods (that can, but do not necessarily, include hybridization methods such as northern blotting) are commonly used for detection of all but the most abundant RNAs, in an effort to overcome the instability of RNA during laboratory manipulations and to amplify the number of copies of RNA to be detected (thereby increasing RNA signal in a relevant assay). For example, reverse transcription/amplification detection approaches, including those that rely on RT-PCR, T₇ RNA polymerase-mediated transcription/amplification, the Van Gelder Eberwine reaction, Qβ replicase amplification, and others are in common use. Several methods during the last few years have, thus, focused on improving the yields of RNA (or DNA) amplicons produced in various amplification methods.

For example, U.S. Pat. No. 5,256,555 to Milburn et al. COMPOSITIONS AND METHODS FOR INCREASING THE YIELDS OF IN VITRO RNA TRANSCRIPTION AND OTHER POLYNUCLEOTIDE SYNTHETIC REACTIONS relates to improved reaction mixtures for in vitro RNA transcription, e.g., using relatively high concentrations of nucleotides (about 12 to 100 mM) as well as optimized concentrations of Mg⁺⁺. The addition of pyrophosphatase is used to prevent inhibitory action of pyrophosphate (a nucleotide polymerization reaction by-product). Similarly, U.S. Pat. No. 6,057,134 to Lader et al. MODULATING THE EFFICIENCY OF NUCLEIC ACID AMPLIFICATION REACTIONS WITH 3′ MODIFIED OLIGONUCLEOTIDES relates to the use of modified primers used in amplification reactions to improve the efficiency and yield of the reactions. Product yield has also been optimized, e.g., by using improved product recovery methods, e.g., as described in U.S. Pat. No. 5,422,241 to Goldrick et al. METHODS FOR HE RECOVERY OF NUCLEIC ACIDS FROM REACTION MIXTURES. These methods avoid the need for protease digestion or organic extraction, relying on selective nuclease digestion of free single stranded RNA followed by precipitation with a precipitating agent. These and many other methods of making nucleic acids, including RNAs, are well described in the literature. See also, Sambrook et al. (2001) Molecular Cloning, A Laboratory Manual 3rd Edition Cold Spring Harbor Laboratory, ISBN: 0879695773 (“Sambrook”) and Ausubel et al., eds., Current Protocols, a joint venture between Greene Publishing Associates, Inc. and John Wiley & Sons, Inc., (supplemented through 2003) (“Ausubel”).

In general, in vitro produced nucleic acids, such as RNA, are produced by solution phase enzymatic synthesis methods. The in vitro enzymatic production of RNA and other nucleic acids is still typically performed in a batchwise fashion. Batchwise production is inherently inefficient, because the expensive components of the assay (e.g., the polymerase(s), transcriptase(s) templates, NTPs, etc.) are thrown out with each batch (that is, the nucleic acid product is purified from the components of the assay, with the assay components being discarded). Moreover, product analysis (e.g., analysis of the relevant nucleic acid amplicon) is ordinarily separated from the production of RNA, adding time and complexity to the overall process of making and using RNA products.

Though not in wide use, continuous flow RNA synthesis methods have also been proposed. For example, U.S. Pat. No. 5,700,667 to Marble et al., STRATEGY FOR THE PRODUCTION OF RNA FROM IMMOBILIZED TEMPLATES, describe a continuous flow bioreactor that uses an immobilized DNA template to enzymatically produce RNA amplicons. The bioreactor consists of an immobilized DNA template coupled to a source of RNA synthesis materials. Reaction products are filtered through an ultrafiltration membrane that retains enzymatic components and the immobilized template, but allows RNA transcripts to pass through the membrane and into the exit stream. This continuous flow system suffers from several drawbacks, however, including the large scale of the reaction (beaker scale), a need for stirring of components during reaction (e.g., due to the scale of the reaction), a lack of integration of the reaction system with product detection systems, and an inability to use the system for diagnostic or other quantitative detection methods. That is, the reactor uses specialized template DNAs, rather than nucleic acids from a biological source to be assayed for a presence, level, or absence of a given nucleic acid of interest. The system is not used for quantifying an unknown biological sample of interest.

The present invention overcomes the difficulties of the prior art in several respects. First, the invention provides a microscale continuous flow bioreactor that produces RNAs in large quantities. The scale of the reactor makes the system suitable for quantitative and qualitative analysis of mRNA from biologically relevant sources, including for expression analysis. These and many other features will become apparent upon a more complete review of the following.

### SUMMARY OF THE INVENTION

The invention comprises microscale bio-reactors and related methods. The bioreactors are configured, e.g., for continuous flow amplification of nucleic acids, including RNA and/or DNA amplification. The use of a continuous flow format provides an advantage to previous methods, in that the concentration of reactants (and products) can be maintained at desired levels, providing for a more controllable and optimizable reaction. For example, because the concentration of reactants can be held at an optimal point by a system, the system can produce optimal amounts of product. Further, because products such as RNA amplicons are continuously removed in certain continuous flow embodiments, amplification reactions do not display product inhibition (inhibition of the reaction by product formation).

In several aspects described herein, RNA amplification provides a particularly desirable constant flow amplification format. Reaction parameters are optimized for the microscale systems, making such micro-scale continuous flow reactions feasible. These constant flow amplification systems can be used in a variety of ways, including for the manufacture of desirable nucleic acids such as RNAs, and for expression analysis. The use of constant flow amplification for quantitative analysis of expressed nucleic acids provides a new general way of performing such analyses.

Accordingly, in a first aspect, the invention provides methods of amplifying at least one nucleic acid such as an RNA. In the methods, one or more RNA amplification reagent is flowed into a chamber, e.g., a microscale reaction chamber. One or more template nucleic acid is amplified to produce one or more RNA amplicon in the microscale reaction chamber, under conditions of constant or semi-constant flow, where one or more reaction parameter is optimized to provide production of the RNA amplicons in the reaction chamber, or flow of the amplicons out of the chamber, or both.

In an alternate embodiment, amplification reactions are performed under conditions of low or no flow, e.g., flowing reaction components are flowed into a chamber, which can be of microscale or non-microscale dimensions, and the reaction is performed for some period of time before additional reactants are flowed into the chamber (and products are flowed out). In either of these embodiments, the reactants and products can be flowed into or out of the chambers through microscale channels, or can be delivered by conventional methods (e.g., manual or robotic pipetting).

In these methods, RNA amplification reagents can include any of a variety of components, e.g., a solid support, the template nucleic acid, a DNA template, a poly(dT) oligonucleotide with an RNA polymerase promoter sequence, a cell, a cell extract, a reverse transcriptase, an rNTP, a dNTP, Mg++, a buffer or buffer component, or the like. Amplifying the nucleic acid can include expressing a DNA that encodes the RNA amplicon, in vitro. Similarly, amplifying can include expressing a plurality of cDNAs that encode total polyA mRNA from a biological sample. The RNA amplification reagents (or one or more additional RNA amplification reagent(s)) can be contained within a bead bed, e.g., that fills a deep portion of a microscale channel. Optionally, the channel has a lateral step up in depth, where the amplification reagents and/or amplicons flow along a side of the bead bed and diffuse laterally into and out of the bead bed.

The biological sample can include biological materials such as cells. Due to the scale of the system, very small numbers of cells can by used, e.g., less than about 10 cells, down to use of a single cell as the source of biological material. Of course, more cells can also be used as the source of biological materials, e.g., where large quantities of amplicon are desired. Thus, about 10 or more, about 100 or more, or even about 1000 or more or 10,000 or more cells can be used.

One feature of the invention is the optimization of reaction parameters, e.g., for performing an mRNA amplification in a microscale system. Such reaction parameters can be any that are relevant to performing the assay, e.g., a rate of flow in the chamber, a temperature in the chamber, a concentration of one or more of the RNA amplification reagents in the chamber, inhibiting or enhancing DNA transcription in the amplification chamber, a channel size leading into or out of the chamber, a size of the chamber, a bead diameter of a bead bound to one or more additional RNA amplification reagent (e.g., a template), total porosity of a bead bed bound to one or more additional RNA amplification reagent, a percent of fluid that diffuses in and out of a bead bed bound to one or more additional RNA amplification reagent as the fluid flows through and along the bead bed, residence time of (and distance traveled by) the reaction substrates or products through the bead bed, direction of flow of one or more reactants or products through a bead bed, and/or the like. In one example embodiment, reactants are flowed through a bead bed (which can be, e.g., in a channel, chamber or well) in a direction transverse (orthogonal) to flow of products out of the bead bed. For example, reactants can be flowed into the bead bed along a long dimension of the bead bed (reactants generally can generally flow relatively freely through a bead bed), while products are flowed across a short dimension of the bead bed (products can be more resistant to flow through the bead bed, and yields can be improved by configuring the flow path of products for reduced flow). Alternately, reagents and products can both be flowed through the short dimension of the bead bed, minimizing trapping of both reagents and products by the bead bed. In either embodiment, the beads themselves are optionally flowed in a direction transverse (orthogonal) to the flow of the reactants and/or products.

Detection of RNA amplicons produced by the microscale bioreactors of the invention is also a feature of the invention. For example, this detection can include detecting the RNA amplicon by flowing the amplicon into contact with an oligonucleotide array. Typically, the RNA amplicons are flowed out of the reaction chamber into contact with the array under constant flow conditions. The array can be in one or more microchamber, or can be a component of a separate device or system. In an alternate embodiment, detecting the RNA amplicon comprises real time detection of RNA amplicon formation, either in a reaction chamber or in a flowing format, e.g., by flowing an aliquot of a labeled amplicon past a fluorescence detector to determine a yield of an amplification. Similarly, the RNA amplicon can be detected by electrophoresing the amplicon through a matrix and detecting at least one resulting size separated RNA amplicon (the RNA is optionally purified and/or fragmented before being size separated, or in an alternate embodiment, before being detected on a nucleic acid array). Detection of the RNA amplicon can be used in any biologically relevant assay, e.g., as a diagnostic or prognostic indicator for one or more polymorphism, SNP, disease or condition. RNA detection optionally includes quantification, e.g., for expression analysis, quality control measurements, or the like.

One application for a microscale bioreactor of the invention, e.g., incorporating amplification and/or nucleic acid detection, is a quality control test of an amplified RNA product, e.g., to determine if it is suitable for use as an input sample for a gene expression array hybridization assay. That is, because DNA microarrays are expensive and the hybridization procedures used in the arrays are time consuming to perform, and because the RNA sample can easily degrade in a short time, it is common practice to perform a quality control assay on amplified RNA (a RNA) before proceeding with the microarray hybridization.

Amplifying the template nucleic acid can include, e.g., performing a Van Gelder-Eberwine series of reactions that converts one or more starting RNA into DNA by reverse transcription, performs a second strand synthesis to produce double stranded DNA and transcribes the double stranded DNA to produce the RNA amplicons. For example, the amplifying step can be performed twice in series, with the RNA amplicon from a first amplification reaction being used as the template nucleic acid for a second amplifying step, which, again, optionally includes performing a Van-Gelder Eberwine reaction.

The RNA amplicon can be further processed, e.g., cleaved or translated into one or more translation products (e.g., one or more proteins). The products of the further processing can be detected in real time. For example, cleavage fragments or translation produces can be detected in real time. One common detection format includes binding an antibody to the product and detecting binding of the antibody to the translation product (e.g., in an ELISA-like assay). In vitro translation reagents can be contacted to the RNA amplicons under conditions of continuous or semi-continuous flow. The RNA amplicon, or DNA copies thereof, can also be cloned into one or more cloning or expression vectors for further amplification or expression studies.

In a related class of embodiments, methods of detecting a presence or absence of one or more target RNA in a biological sample are provided. In the methods, one of more reverse transcription reaction is performed on sample RNA (e.g., by contacting the sample RNA with a reverse transcriptase) from the biological sample to produce one or more cDNA. Transcription reagents are flowed into contact with the one or more cDNA, and one or more expression reaction is performed on the cDNA under conditions of continuous or semi-continuous flow. Detection of one or more RNA amplicon, or detection of a lack of the amplicon, produced by the expression reaction, provides an indicator of the presence or absence of the target RNA. Features of the embodiments described above can be applied to this class of methods as well, e.g., with respect to RNA amplification reagents, sources of biological materials, optimization of reaction parameters, detection of RNA amplicons, use of the Van Gelder-Eberwine reactions, RNA amplicon processing, and/or the like.

The target RNA can be, e.g., an mRNA such as a polyA mRNA, e.g., comprising one or more polymorphism (e.g., one or more SNP). In one typical embodiment, the target RNA is isolated from a biological sample that comprises one or more cell. Similarly, the sample RNA can include, e.g., a plurality of mRNA transcripts from the biological sample, e.g., total poly A mRNA from the biological sample.

The cDNA is optionally coupled to a solid support prior to said flowing. For example, the solid support can be a surface in a channel of a microscale device, and/or can include one or more particles (e.g., polymer, metal, ceramic, silica or glass beads). Particles can be flowed into one or more microscale chamber where the transcription reagents are flowed into contact with said particles. In one embodiment, the cDNA is optionally incorporated into a gel.

The expression reaction can be performed in a microfluidic chamber (e.g., channel, compartment, well or the like). The expression reaction optionally includes optimizing one or more reaction parameter relevant to performing the reaction in such as system, e.g., optimizing a rate of flow in the chamber, a temperature in the chamber, a concentration of one or more of the RNA amplification reagents in the chamber, inhibiting or enhancing DNA transcription in the amplification chamber, a channel size leading into or out of the chamber, and a size of the chamber, a bead diameter of a bead bound to one or more additional RNA amplification reagent, total porosity of a bead bed bound to one or more additional RNA amplification reagent, a percent of fluid that diffuses in and out of a bead bed bound to one or more additional RNA amplification reagent as the fluid flows through and along the bead bed, and/or the like. Optionally, the expression reaction comprises directly transcribing the cDNA. In another embodiment, performing the expression reaction comprises indirectly transcribing the cDNA. Detecting the RNA amplicon optionally includes flowing the RNA amplicon into contact with an oligonucleotide array. For example, the RNA amplicon can be flowed out of a reaction chamber in which the expression reaction is performed and into contact with the array, under constant flow conditions. The array can be in one or more microchamber, or can be separate from a microfluidic system used to make the amplicon.

The RNA amplicon can include real time detection and/or quantification of RNA amplicon formation, and/or can include detecting or quantifying amplicon formation after amplification. For example, in one aspect, detecting the RNA amplicon comprises electrophoresing the amplicon through a matrix and detecting at least one size separated RNA amplicon. Optionally, the matrix is present in a microchannel fluidly coupled to a microchamber in which the expression reaction is performed. Detecting the RNA amplicon can be, e.g., a diagnostic or prognostic indicator for or one or more polymorphism, SNP, disease or condition.

The RNA amplicon is optionally detected by translating the amplicon into one or more translation product (e.g., protein) and detecting the translation product. Optionally, as with other detection steps, the translation reagent can be contacted to the RNA amplicon under conditions of continuous or semi-continuous flow. Similarly, the RNA amplicon can be processed by any available method (cleavage, cloning, reverse transcription and cleavage or cloning, etc.) and detected using appropriate detection methods.

The features of the above classes of methods can be combined and/or substituted for one another.

In addition to the above methods, the invention also provides systems for making one or more RNA. The systems include, e.g., a microchamber comprising one or more template nucleic acid, one or more source of one or more transcription reaction component, and a flow controller that directs continuous or semi-continuous flow of the one or more transcription reaction component into the microchamber. The rate of flow is optimized for flow of the one or more transcription reaction component into the chamber, flow of RNA amplicons out of the chamber, amplification of RNA within the chamber, or a combination thereof. The various features noted above with respect to the methods, e.g., with respect to types of template nucleic acid, sources and types of biological materials, transcription reaction components, translation reaction components, and the like, can be used in or incorporated into the systems of the invention.

The flow controller optionally comprises a positive or negative (vacuum) pressure source, or an electrokinetic flow component, or both. For example, in one aspect, both electrokinetic and pressure based flow are used, with the application of an electric field resulting in quick movement of charged nucleic acid amplicons, such as RNAs, through a bead bed or other structure in the microscale device, while pressure based flow is used for general reagent control. Optionally, the flow controller directs continuous or semi continuous flow of RNA amplicons out of the microchamber and into an additional microchamber or microchannel. The flow controller is optionally configured to optimize one or more reaction parameter as noted above, e.g., a rate of flow in the microchamber, a concentration of one or more of the RNA amplification reagent in the microchamber, inhibiting or enhancing DNA transcription in the microchamber, etc.

Typically, the system includes a detector that detects an RNA amplicon in the microchamber or in a channel or chamber fluidly coupled to the microchamber. Optionally, the detector comprises an oligonucleotide array or relevant detection element as noted above with respect to the discussion of the methods of the invention. The detector typically further comprises detection optics (or other suitable detector elements, such as scintillation counters or the like) which detect one or more signals from the array. In one typical embodiment, the detector comprises or is operably coupled to a computer that comprises instructions for determining whether a signal detected by the detector corresponds to an RNA of interest. As with the methods above, the RNA of interest can comprise, e.g., a polymorphism, such as a SNP, or can be a diagnostic or prognostic indicator for a disease or condition.

Optionally, the system includes a temperature controller that controls temperature in the microchamber. These can include resistive heaters, Peltier heating/cooling elements, refrigeration systems, electrodes for joule heating of fluid, e.g., in microchannels, temperature blocks, water baths or other appropriate heating and/or cooling components, as well as controller elements such as temperature detectors, voltage controllers, etc.

The system can also include system instructions, e.g., embodied in system software (e.g., in a computer or computer readable medium comprised within or operably linked to the system). These system instructions can direct the system to perform any of the method steps noted above, e.g., in one embodiment, the system includes instructions for performing a Van Gelder-Eberwine series of reactions that converts one or more starting RNA into DNA by reverse transcription, performs a second strand synthesis to produce double stranded DNA and transcribes the double stranded DNA to produce the RNA amplicons. For example, the instructions can direct the various temperature and flow controllers to flow appropriate materials from sources to reaction sites and/or to control reaction conditions (e.g., temperature) at the sites.

In a related embodiment, the invention provides an expression profiling system. The system includes a chamber comprising cDNA corresponding to a plurality of mRNAs from a biological sample, a source of an in vitro transcription reagent, a flow controller configured to continuously or semi-continuously flow the in vitro transcription reagents from the source of in vitro transcription reagent to the chamber, a detector configured to detect amplified RNA transcribed from the cDNA, and, instructions for correlating signals detected by the detector to one or more of the plurality of mRNAs. The above system and method components can be applied to this embodiment as well. For example, the plurality of mRNAs optionally comprises total poly A mRNA from the biological sample. The detector and temperature controller configurations of the preceding class of system embodiments can also be applied to the present system.

In one useful embodiment, the system comprises instructions for correlating the signals to a complete mRNA expression profile for the biological sample. This provides an expression profile of a sample of interest, which is extremely useful in determining mRNA function, in diagnosing disease, in monitoring an effect of a drug, and, e.g., in pharmacogenomic studies of a tissue, individual or population.

The chamber is optionally a microfluidic chamber and the flow is controller is optionally configured to optimize one or more of: flow of the in vitro transcription reagent into the chamber, flow of RNA amplicons out of the chamber, amplification of RNA within the chamber, or a combination thereof.

The expression profiling system optionally includes a source of in vitro transcription or translation reagent(s) fluidly coupled to the chamber, e.g., where the flow controller is configured to continuously or semi-continuously flow the in vitro transcription/translation reagent into contact with the RNA transcribed from the cDNA. Similar to the preceding class of system embodiment, the system can include instructions for practicing any of the method steps noted above. For example, in one embodiment, the system includes system instructions for performing a Van Gelder-Eberwine series of reactions that converts one or more starting RNA into DNA by reverse transcription, performs a second strand synthesis to produce double stranded DNA and transcribes the double stranded DNA to produce RNA amplicons which are detected by the detector.

The invention also includes products produced by the methods herein, systems that comprise such products and kits. The kits can include, e.g., any of the system components noted herein, or any component useful in a method herein, e.g., packaged in appropriate containers or other packaging material, optionally in combination with instructions for practicing the methods herein, or for assembling or using the systems herein.

### BRIEF DESCRIPTION OF THE FIGURES

FIG. 1 schematically illustrates an example bioreactor device comprising microscale channels, reagent wells, waste wells and heating and cooling zones.

FIG. 2 schematically illustrates an example bioreactor device of the invention comprising multiple reaction channels.

FIG. 3 is an electron photomicrograph of a bead bed laid down in one of the reaction channels of the device of FIG. 2.

FIG. 4 schematically illustrates a computer model of heat transfer and the resulting isothermal counters as shown for the device of FIGS. 2-3.

FIG. 5 schematically illustrates an example microscale bioreactor comprising multiple input and output wells, reagent wells including a bead well and 4 reaction chambers.

FIG. 6 schematically illustrates a variation of the device of FIG. 5 that comprises 4 additional wells.

FIG. 7 schematically illustrates an example system of the invention.

FIG. 8 shows data from an experimental amplification and purification.

FIG. 9 shows data from an experimental amplification and purification (1 data plot, with a peak showing an aRNA product highlighted.

FIG. 10 shows data from an experimental amplification and purification (two data plots), illustrates the results of amplifying RNA from total RNA.

FIG. 11 schematically illustrates a design for a microscale bioreactor.

FIG. 12 shows data for an on device experiment performed with a device according to FIG. 11, showing 0.6 μL of 250 ng/μL amplified RNA being collected out of a 1000 bead bead-bed.

FIG. 13 shows data for an additional experiment, in which about 140 beads were loaded into the bead bed.

FIG. 14 shows data for a typical reaction run according to standard protocols for RNA amplification using a MessageAmp™ kit (available from Ambion), run on mouse RNA.

FIG. 15 schematically illustrates a microscale bioreactor comprising a main channel and a transverse channel network for delivery of reagents and removal of products.

FIG. 16 schematically illustrates the reaction chamber of the device of FIG. 15.

FIG. 17 schematically illustrates an alternate microscale bioreactor comprising a main channel and a transverse channel network for delivery of reagents and removal of products.

FIG. 18 schematically illustrates a system of the invention.

### DEFINITIONS

The following definitions are directed to the current application and are not to be imputed to any related or unrelated case, e.g., to any commonly owned patent or application.

Before describing the present invention in detail, it is to be understood that this invention is not limited to particular systems or methods, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the context clearly dictates otherwise. Thus, for example, reference to “a substrate” optionally includes a combinations of two or more substrates; reference to an “RNA” optionally includes a plurality of RNAs, and/or the like.

A nucleic acid is “amplified” when one or more copies of the nucleic acid, or at least one strand thereof, are copied and/or transcribed. Thus, a DNA can be amplified to produce DNAs or RNAs, and RNA can be amplified by transcribing it from DNA (which, itself, can be made by reverse transcription of an RNA), etc.

An “RNA amplification reagent” is a reagent that participates in or facilitates amplification of a given RNA. These can include enzymes, nucleotides, buffers, fluids or any other relevant reagent.

A “template nucleic acid” is a nucleic acid that is to be copied or transcribed.

A “microscale chamber” is a cavity having at least one dimension that is about 500 μM or less, and can be as little as 100 μM or less, 10 μM or less, 1 μM or less or 0. 1 μM or less. The chamber can be a microfluidic channel, a well or the like. The chamber can be fully enclosed or open on one or more sides or at one or more end points.

An “amplicon” is a molecule made by copying or transcribing another molecule, e.g., as occurs in PCR, transcription, and/or cloning.

“Constant or semi-constant” flow involves flow that is not stopped for a significant period of time during the reaction or assay that the flow is applied to. It is, of course, understood that flow may be stopped before or after the reaction or assay at issue in a continuous flow assay.

An “array” is an assemblage of elements. The assemblage can be spatially ordered (a “patterned array”) or disordered (a “randomly patterned” array). The array can form or comprise one or more functional elements (e.g., a probe region on a microarray) or it can be non-functional.

A “translation product” is a product (typically a polypeptide) produced as a result of the translation of a nucleic acid. A “transcription product” is a product (e.g., an RNA, optionally including mRNA, or, e.g., a catalytic or biologically active RNA) produced as a result of transcription of a nucleic acid.

### Detailed Description

The present invention is directed towards methods and systems for nucleic acid (including RNA or DNA) analysis. These systems and methods typically utilize constant or semi-constant flow of reactants into and products out of a microscale reaction chamber (though non-constant flow applications are also discussed herein). The nucleic acid amplification reagents are flowed into the chamber under flow conditions optimized to the microscale reaction. The reactants flow through the chamber under conditions (e.g., with respect to flow, temperature, reaction volume, chamber dimensions, concentration, ionic strength (e.g., MgCl₂⁺⁺ strength, etc.) that are optimized for amplification in the chamber and flow of products out of the chamber. One of skill will recognize that overproduction of products in the system can be as undesirable as underproduction, in that overproduction of product can result in clogging of the microscale system (of course, underproduction results in decreased yield of products). This invention finds use in a variety of contexts, including for gene expression analysis, quality control analysis, SNP detection and others.

One advantage of continuous flow systems is that continuous flow provides a mechanism for keeping product and reactant concentrations constant. This has several clear advantages. First, reactant concentration can be optimized for the system at issue, and then held at that optimized concentration. This is in contrast to a standard stopped flow reaction, in which reactants are typically put into the reaction at a concentration higher than optimal and the reaction is run to completion (where the reactant concentration is lower than optimal). Similarly, product inhibition is a significant problem in nucleic acid amplification reactions. That is, in addition to the typical reaction inhibition by product formation due to mass action effects, nucleic acid amplification products pose special problems in amplification reactions. That is, the products often inhibit product formation, e.g., due to binding of the products to template materials in the reaction. For example, inhibitory RNA (RNAi) effects are common, resulting in transcription suppression, through both sense and anti-sense mechanisms, in vitro and in vivo. In the constant flow systems of the invention, these product reaction inhibition effects can be reduced by constantly flowing product nucleic acids (e.g., aRNAs) out of the system.

An additional feature of the invention is the use of various quantitative amplification methods in the microscale systems, such as the Van Gelder-Eberwine reaction, to produce nucleic acid amplicons (e.g., RNAs, DNAs, cDNAs, etc.) that are representative of the concentrations and quantities of nucleic acids present in a starting sample to be amplified. This permits quantitative determination of the relative rations of such starting nucleic acid materials. One particularly useful feature of this embodiment is that such determinations provide for expression analysis of nucleic acid transcripts in biological starting materials such as cells or tissues. Expression analysis provides a basic tool for diagnosis of disease, analysis of environmental or drug effects on cellular expression of transcripts (a common test performed when screening libraries of compounds for molecules that modify transcription of nucleic acids) and the like.

Thus, the presence or absence of any nucleic acid of interest in a biological sample can be determined (e.g., as is commonly performed for polymorphism analysis, e.g., single-nucleotide polymorphism or “SNP” analysis), as can the relative level of any nucleic acid, as is commonly performed in expression analysis studies. The present invention permits the synthesis of nucleic acids, including RNAs, for the detection of the nucleic acids, determination of their relative levels in a starting sample of nucleic acid, and for their use as probes or other diagnostic reagents (e.g., capture agents) in any suitable nucleic acid assay.

An additional aspect of the invention is that such systems are conveniently integratable with other microscale (or non-microscale) components or systems. For example, the microscale systems can include translation reagents that translate nucleic acid amplicons, producing proteins of interest. This can also be performed in a quantitative way; accordingly, such produced proteins can be used in any suitable assay to monitor expression of nucleic acids, or for any other purpose that proteins of interest are typically used for, including, e.g., as diagnostic reagents, as therapeutic reagents, and/or the like. Similarly, the microscale systems can include or be operably linked to detection systems such as nucleic acid arrays, protein analysis systems, electrophoretic nucleic acid analysis systems, or the like.

Alternatively, nucleic acids or proteins produced by the microscale systems of the invention can be harvested from the systems and used in any available system or method that uses such components. For example, RNA amplicons can be harvested and hybridized to an array of nucleic acid probes, e.g., for expression monitoring, SNP determination or the like. Similarly, such amplified nucleic acids can be sequenced using available sequencing methods and/or systems, analyzed by restriction analysis, electrophoresis or the like. Proteins made in the systems of the invention can be analyzed by western blotting, hybridization to antibody or ligand arrays, ELISA analysis or any other method.

One application of the invention is for quality control applications. Because one can obtain amplified nucleic acids from the systems of the invention (at essentially any time point prior to, during or after an amplification) the nucleic acids can be checked by any of a variety of quality control approaches using the methods herein. These include length determination, detection of particular sequences, and the like. For example, cDNA can be labeled by available method(s), e.g., by reverse transcription and label incorporation into the DNA. DNA made from an initial RNA sample can be assayed for any quality control feature(s) before RNA amplification (e.g., before an in vitro transcription step). Alternately resulting amplified RNA can be directly assayed, e.g., by fragmenting and, if desired, cleaning up (e.g., purifying or partially purifying) the amplified RNA. The amplified RNA can be, e.g., concentrated, purified, fragmented, isolated, etc. Quality control is generally useful as an approach for determining whether an amplification reaction is proceeding as expected.

Nucleic Acids and Samples of Interest

The nucleic acid of interest to be amplified, transcribed, translated and/or detected in the methods of the invention can be essentially any nucleic acid. The sequences for many nucleic acids and amino acids (from which nucleic acid sequences can be derived via reverse translation) are available. No attempt is made to identify the millions of known nucleic acids, any of which can be detected in the methods of the invention. Common sequence repositories for known nucleic acids include GenBank® EMBL, DDBJ and the NCBI. Other repositories can easily be identified by searching the internet. The nucleic acid to be amplified, transcribed, translated and/or detected can be an RNA (e.g., where amplification includes RT-PCR or LCR, the Van-Gelder Eberwine reaction or Ribo-SPIA) or DNA (e.g., cDNA or genomic DNA), or even any analogue thereof (e.g., for detection of synthetic nucleic acids or analogues thereof, where the sample of interest includes artificial nucleic acids). Any variation in a nucleic acid can be detected, e.g., a mutation, a polymorphism, a single nucleotide polymorphism (SNP), an allele, an isotype, etc. Further, because the present invention produces quantitative amplification, if desired, one can detect variation in expression levels or gene copy numbers by the methods herein.

For example, the methods of the invention are useful in screening samples derived from patients for a nucleic acid of interest, e.g., from bodily fluids (blood, urine etc.), tissue, and/or waste from the patient. Thus, stool, sputum, saliva, blood, lymph, tears, sweat, urine, vaginal secretions, ejaculatory fluid or the like can easily be screened for nucleic acids by the methods of the invention, as can essentially any tissue of interest. These samples are typically taken, following informed consent, from a patient by standard medical laboratory methods.

Prior to aliquotting and amplification, nucleic acids are optionally purified from the samples by any available method, e.g., those taught in Berger and Kimmel, Guide to Molecular Cloning Techniques Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”); and/or Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). A plethora of kits are also commercially available for the purification of nucleic acids from cells or other samples (see, e.g., EasyPrep™, FlexiPrep™, both from Pharmacia Biotech; StrataClean™, from Stratagene; and, QIAprep™ from Qiagen). Alternately, samples can simply be directly subjected to amplification, e.g., following aliquotting and dilution.

One class of nucleic acids of interest to be detected in the methods herein are those involved in cancer. Any nucleic acid that is associated with cancer can be detected in the methods of the invention, e.g., those that encode over expressed or mutated polypeptide growth factors (e.g., sis), over expressed or mutated growth factor receptors (e.g., erb-B1), over expressed or mutated signal transduction proteins such as G-proteins (e.g., Ras), or non-receptor tyrosine kinases (e.g., abl), or over expressed or mutated regulatory proteins (e.g., myc, myb, jun, fos, etc.) and/or the like. In general, cancer can often be linked to signal transduction molecules and corresponding oncogene products, e.g., nucleic acids encoding Mos, Ras, Raf, and Met; and transcriptional activators and suppressors, e.g., p53, Tat, Fos, Myc, Jun, Myb, Rel, and/or nuclear receptors. p53, colloquially referred to as the “molecular policeman” of the cell, is of particular relevance, as about 50% of all known cancers can be traced to one or more genetic lesion in p53.

Taking one class of genes that are relevant to cancer as an example for discussion, many nuclear hormone receptors have been described in detail and the mechanisms by which these receptors can be modified to confer oncogenic activity have been worked out. For example, the physiological and molecular basis of thyroid hormone action is reviewed in Yen (2001) “Physiological and Molecular Basis of Thyroid Hormone Action” Physiological Reviews 81(3):1097-1142, and the references cited therein. Known and well characterized nuclear receptors include those for glucocorticoids (GRs), androgens (ARs), mineralocorticoids (MRs), progestins (PRs), estrogens (ERs), thyroid hormones (TRs), vitamin D (VDRs), retinoids (RARs and RXRs), and the peroxisome proliferator activated receptors (PPARs) that bind eicosanoids. The so called “orphan nuclear receptors” are also part of the nuclear receptor superfamily, and are structurally homologous to classic nuclear receptors, such as steroid and thyroid receptors. Nucleic acids that encode any of these receptors, or oncogenic forms thereof, can be detected in the methods of the invention. About 40% of all pharmaceutical treatments currently available are agonists or antagonists of nuclear receptors and/or oncogneic forms thereof, underscoring the relative importance of these receptors (and their coding nucleic acids) as targets for analysis by the methods of the invention.

One class of nucleic acids of interest are those that are diagnostic of colon cancer, e.g., in samples derived from stool. Colon cancer is a common disease that can be sporadic or inherited. The molecular basis of various patterns of colon cancer is known in some detail. In general, germline mutations are the basis of inherited colon cancer syndromes, while an accumulation of somatic mutations is the basis of sporadic colon cancer. In Ashkenazi Jews, a mutation that was previously thought to be a polymorphism may cause familial colon cancer. Mutations of at least three different classes of genes have been described in colon cancer etiology:oncogenes, suppressor genes, and mismatch repair genes. One example nucleic acid encodes DCC (deleted in colon cancer), a cell adhesion molecule with homology to fibronectin. An additional form of colon cancer is an autosomal dominant gene, hMSH2, that comprises a lesion. Familial adenomatous polyposis is another form of colon cancer with a lesion in the MCC locus on chromosome #5. For additional details on Colon Cancer, see, Calvert et al. (2002) “The Genetics of Colorectal Cancer” Annals of Internal Medicine 137 (7): 603-612 and the references cited therein. For a variety of colon cancers and colon cancer markers that can be detected in stool, see, e.g., Boland (2002) “Advances in Colorectal Cancer Screening: Molecular Basis for Stool-Based DNA Tests for Colorectal Cancer: A Primer for Clinicans” Reviews In Gastroenterological Disorders Volume 2, Supp. 1 and the references cited therein.

Cervical cancer is another preferred target for detection, e.g., in samples obtained from vaginal secretions. Cervical cancer can be caused by the papova virus and has two oncogenes, E6 and E7. E6 binds to and removes p53 and E7 binds to and removes PRB. The loss of p53 and uncontrolled action of E2F/DP growth factors without the regulation of pRB is one mechanism that leads to cervical cancer.

Another preferred target for detection by the methods of the invention is retinoblastoma, e.g., in samples derived from tears. Retinoblastoma is a tumor of the eyes which results from inactivation of the pRB gene. It has been found to transmit heritably when a parent has a mutated pRB gene (and, of course, somatic mutation can cause non-heritable forms of the cancer).

Neurofibromatosis Type 1 can be detected in the methods of the invention. The NF1 gene is inactivated, which activates the GTPase activity of the ras oncogene. If NF1 is missing, ras is overactive and causes neural tumors. The methods of the invention can be used to detect Neurofibromatosis Type 1 in CSF or via tissue sampling.

Many other forms of cancer are known and can be found by detecting associated genetic lesions using the methods of the invention. Cancers that can be detected by detecting appropriate lesions include cancers of the lymph, blood, stomach, gut, colon, testicles, pancreas, bladder, cervix, uterus, skin, and essentially all others for which a known genetic lesion exists. For a review of the topic, see, The Molecular Basis of Human Cancer Coleman and Tsongalis (Eds) Humana Press; ISBN: 0896036340; 1st edition (August 2001).

Similarly, nucleic acids from pathogenic or infectious organisms can be detected by the methods of the invention, e.g., for infectious fungi, e.g., Aspergillus, or Candida species; bacteria, particularly E. coli, which serves a model for pathogenic bacteria (and, of course certain strains of which are pathogenic), as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), (−) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

A variety of nucleic acid encoding enzymes (e.g., industrial enzymes) can also be detected according to the methods herein, such as amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases. Similarly, agriculturally related proteins such as insect resistance proteins (e.g., the Cry proteins), starch and lipid production enzymes, plant and insect toxins, toxin-resistance proteins, Mycotoxin detoxification proteins, plant growth enzymes (e.g., Ribulose 1,5-Bisphosphate Carboxylase/Oxygenase, “RUBISCO”), lipoxygenase (LOX), and Phosphoenolpyruvate (PEP) carboxylase can also be detected.

Nucleic Acid Amplification

One of the most powerful and basic technologies for nucleic acid detection is nucleic acid amplification. That is, in many typical formats, such as the polymerase chain reaction (PCR), reverse-transcriptase PCR (RT-PCR), ligase chain reaction (LCR), and Qp replicase and other RNA/transcription mediated techniques (e.g., NASBA), amplification of a nucleic acid of interest precedes detection of the nucleic acid of interest, because it is easier to detect or manipulate many copies of a nucleic acid than it is a single copy. The present invention improves upon existing methods of amplification by providing continuous flow amplification reactions in a microscale system for making nucleic acid amplicons, including RNA amplicons.

In the methods of the invention, reagents appropriate for performing amplification reactions are introduced into a microscale reaction chamber, typically under conditions optimized to favor amplicon production in the microscale system. These reagents can be any of those typically used for nucleic acid amplification, e.g., for performing PCR, LCR, Qβ-replicase amplification, Van Gelder-Eberwine amplification, or the like.

These amplification methods can include various polymerase or ligase mediated amplification methods, such as PCR or LCR, in vitro transcription, and/or the like. PCR, RT-PCR and LCR are in particularly broad use, in many different fields. Details regarding the use of these and other amplification methods can be found in any of a variety of standard texts, including, e.g., :Sambrook, Ausubel, and PCR Protocols A Guide to Methods and Applications (Innis et al. eds) Academic Press Inc. San Diego, Calif. (1990) (Innis). Many available biology texts also have extended discussions regarding PCR and related amplification methods. Additional details regarding nucleic acid amplification can be found in Mullis et al., (1987) U.S. Pat. No. 4,683,202; Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995). Improved methods of amplifying large nucleic acids by PCR are summarized in Cheng et al. (1994) Nature 369: 684-685, and the references therein.

Sample-specific methods of performing amplification are also well known and can be found in the preceeding references. That is, amplification protocols and sample preparation methods can vary, depending on the sample of interest, and the literature provides considerable details in this respect. For example, details regarding amplification of nucleic acids in plants can be found, e.g., in Plant Molecular Biology (1993) Croy (ed.) BIOS Scientific Publishers, Inc. Similarly, additional details regarding amplifications for cancer detection can be found in any of a variety of sources, e.g., Bernard and Wittwer (2002) “Real Time PCR Technology for Cancer Diagnostics Clinical Chemistry 48(8):1178-1185; Perou et al. (2000) “Molecular portraits of human breast tumours” Nature 406:747-52; van't Veer et al. (2002) “Gene expression profiling predicts clinical outcome of breast cancer” Nature 415:530-6; Rosenwald et al. (2001) “Relation of gene expression phenotype to immunoglobulin mutation genotype in B cell chronic lymphocytic leukemia” J Exp Med 194:1639-47; Alizadeh et al. (2000) “Distinct types of diffuse large B-cell lymphoma identified by gene expression profiling” Nature 403:503-11; Garber et al. (2001) “Diversity of gene expression in adenocarcinoma of the lung” Proc Natl Acad Sci USA 98: 13784-9; Tirkkonen et al. (1998) “Molecular cytogenetics of primary breast cancer by CGH” Genes Chromosomes Cancer 21:177-84; Watanabe et al. (2001) “A novel amplification at 17q21-23 in ovarian cancer cell lines detected by comparative genomic hybridization” Gynecol Oncol 81:172-7, and many others.

One of skill will also appreciate that essentially any RNA can be converted into a double stranded DNA suitable for restriction digestion, PCR or LCR amplification, and/or downstream manipulations (such as sequencing or cloning), e.g., using reverse transcriptase and a polymerase. See, Ausubel, Sambrook and Berger, all supra.

RT PCR/Transcription

RT-PCR is a common procedure that results in the production of DNA amplicons that correspond to RNAs (typically mRNAs) used as original template materials. The method can be performed according to the present invention by flowing the appropriate RT PCR reagents into contact in a microscale reaction chamber, e.g., in an order according to the relevant method, under conditions optimized for microscale reactions (e.g., with respect to temperature, flow rates, reactant concentrations, and the like). In brief, the protocol includes contacting an mRNA with a reverse transcriptase, in the presence of an appropriate extendible primer (e.g., a poly T oligonucleotide, that binds, e.g., a polyA tail of a typical mRNA). The reverse transcriptase copies the mRNA into a first cDNA strand, which is then replicated according to standard PCR methods. See also, Innis, Sambrook and Ausubel, supra.

Van Gelder Eberwine

A variant of RT-PCR that produces an RNA amplicon is a T7-based (linear) amplification method first developed by Van Gelder, Eberwine and coworkers, commonly referred to as the “Van Gelder-Eberwine reaction.” As with the other reactions herein, the method is adapted to the present invention by flowing appropriate reagents into contact in an appropriate microscale chamber, e.g., under temperature, concentration, flow and other reaction conditions optimized for the reaction in the microscale system.

The Van Gelder-Eberwine reaction uses a synthetic poly(dT) primer containing a phage T7 RNA polymerase promoter to prime synthesis of first strand cDNA by reverse transcription of poly(A) RNA (a common form of mRNA). Second strand cDNA is synthesized by degrading the poly(A) RNA strand with RNase H, followed by synthesis with E. coli DNA polymerase I. Amplified antisense RNA (aRNA) is obtained from in vitro transcription of the double-stranded cDNA (ds cDNA) template using T7 RNA microarrays. See also, Van Gelder et al. (1990) “Amplified RNA synthesized from limited quantities of heterogeneous cDNA” Proc Natl Acad Sci USA 87:1663-1667; Eberwine et al. (1992) “Analysis of gene expression in single live neurons” Proc Natl Acad Sci USA 89:3010-3014; Phillips and Eberwine (1996) “Antisense RNA amplification: a linear amplification method for analyzing the mRNA population from single living cells” Methods 10:283-288; and Zhao et al. (2002) “Optimization and evaluation of T7 based RNA linear amplification protocols for cDNA microarray analysis” BMC Genomics 3:31. Kits for practicing the Van Gelder Eberwine protocol are commercially available, e.g., from Ambion, Inc. (Austin Tex.). An advantage of the Van Gelder Eberwine protocol over standard RT-PCR amplification methods is that it provides representative (linear) amplification, making quantification of starting materials (e.g., relative numbers of source RNAs) possible from detection and quantification of RNA products. This finds use in the present invention in using one or more Van Gelder-Eberwine reactions to monitor gene expression (e.g., when performing expression monitoring).

Ribo-SPIA RNA Amplification

Ribo-SPIA, like the Van Gelder Eberwine approach, is a linear amplification process that generates “antisense” cDNA by DNA replication of a double stranded cDNA that is prepared by reverse translation of an RNA starting template. As with other methods herein, the method is adapted to the present invention by flowing appropriate reagents for Ribo-SPIA into contact in an appropriate microscale chamber, e.g., under flow and other reaction conditions optimized for the reaction in the microscale system.

Ribo-SPIA uses a DNA polymerase, a DNA/RNA primer and RNAse H in a homogeneous isothermal reaction that provides amplification of DNA sequences. That is, an initial RNA template is amplified using the chimeric DNA/RNA primer, to produce single stranded cDNA. DNA polymerase produces double stranded DNA. RNAse H cleaves the chimeric primer and the polymerase initiates from a fresh primer on the original template. The resulting linearly amplified cDNA is complementary to the original cDNA. Because this is a linear process, it, like the Van Gelder Eberwine protocol, results in representative expression, making it possible to correlate quantification of the cDNA product with the mRNA starting material. Commercial kits for performing Ribo-SPIA are available from NuGen Technologies (San Carlos, Calif.), e.g., the Ovation SPIA™ kit. See also, WO 02/072772 and US 2003/0017591 A1. As with the Van Gelder-Eberwine reaction, this approach finds use in the present invention, e.g., in performing expression monitoring reactions.

Upstream Processing

Prior to amplification, a sample of interest can be processed, e.g., purified, aliquotted and/or diluted using standard or microfluidic sample processing approaches (or combinations thereof). For example, standard fluid handling approaches for dilution/aliquotting include, e.g., pipetting appropriate volumes of the sample into microtiter trays and adding an appropriate diluent. These operations can be performed manually or using available high throughput fluid handlers that are designed to use microtiter trays. High throughput equipment (e.g., incorporating automated pipettors and/or robotic microtiter tray handling) can be used, e.g., where the present invention includes making and using several aliquots of a sample of interest, or several samples.

Many automated systems for fluid handling are commercially available and can be used for aliquotting and/or diluting a sample in the context of the present invention. For example, a variety of automated systems are available from the Zymark Corporation (Zymark Center, Hopkinton, Mass.), which utilize various Zymate systems (see also, http://www.caliperls.com/), which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). In any case, conventional high throughput systems can be used in place of, or in conjunction with microfluidic systems (for example, conventional systems can be used to aliquot samples into microtiter trays, from which microfluidic systems can draw materials) in practicing the methods of the invention.

Microfluidic systems provide a preferred fluid handling and amplification technology that can conveniently be applied to the present invention. In typical embodiments, samples are drawn into microfluidic devices that comprise networks of microscale cavities (channels, chambers, etc., having at least one dimension less than about 500 μM in size and often less than about 100 μM) and the samples are mixed, diluted, purified, aliquotted or otherwise manipulated in the network of cavities. For example, the microscale device can comprise one or more capillary, in fluid communication with the network, extending outward from a body structure of the microscale device. Negative pressure (vacuum) is applied to the capillary and fluids are drawn into the network from a container (e.g., a well on a microtiter tray). Alternately, positive pressure can also be used to push fluids into the capillary or microchannel network. This process can be multiplexed by using a device that comprises multiple capillary channels, permitting many samples to be drawn into the network and processed simultaneously. Sample interfaces with dried samples can also be performed using this basic system, e.g., by expelling fluid from the capillary to hydrate samples prior to drawing them into the microfluidic device. For either approach, see also, U.S. Pat. No. 6,482,364 to Parce, et al. (Nov. 19, 2002) MICROFLUIDIC SYSTEMS INCLUDING PIPETTOR ELEMENTS; U.S. Pat. No. 6,042,709 to Parce, et al. (Mar. 28, 2000) MICROFLUIDIC SAMPLING SYSTEM AND METHODS; U.S. Pat. No. 6,287,520 to Parce, et al. (Sep. 11, 2001) ELECTROPIPETTOR AND COMPENSATION MEANS FOR ELECTROPHORETIC BIAS and U.S. Pat. No. 6,235,471 to Knapp, et al. (May 22,2001) CLOSED-LOOP BIOCHEMICAL ANALYZERS. Essentially any fluid manipulation (aliquotting, purifying, diluting, heating and/or cooling) can be performed in the network using available methods. Details regarding dilution and aliquotting operations in microscale devices can be found in the patent literature, e.g., U.S. Pat. No. 6,149,870 to Parce, et al. (Nov. 21, 2000) APPARATUS FOR IN SITU CONCENTRATION AND/OR DILUTION OF MATERIALS IN MICROFLUIDIC SYSTEMS; U.S. Pat. No. 5,869,004 to Parce, et al. (Feb. 9, 1999) METHODS AND APPARATUS FOR IN SITU CONCENTRATION AND/OR DILUTION OF MATERIALS IN MICROFLUIDIC SYSTEMS; and U.S. Pat. No. 6,440,722 to Knapp, et al. (Aug. 27, 2002) MICROFLUIDIC DEVICES AND METHODS FOR OPTIMIZING REACTIONS. Samples and components to be mixed/diluted or aliquotted can be brought into the microscale device through pipettor elements or from reaction component reservoirs on the device itself, or, commonly, both. For example, the sample can be brought into the microfluidic device through a pipettor channel and diluted and supplied with common reagents from an on device dilution and/ or reagent reservoir(s). Locus specific reagents (e.g., amplification primers, e.g., for use in a Van Gelder-Eberwine reaction) can be on the device in wells, or stored off the device, e.g., in microtiter plates (in which case they can be accessed by the pipettor channel). Any or all of these operations can be performed in a continuous or stopped flow format, though the continuous flow format is typically preferred herein.

Commercial systems that perform all aspects of fluid handling and analysis that can be used in the practice of the present invention are available. Examples include the LabChip® 3000 HTS system and the LabChip® 90 system from Caliper Life Sciences, Inc. (Mountain View, Calif.). These systems performs experiments in serial, continuous flow fashion and employ a “chip-to-world” interface, or sample access system, called a sipper through which materials in microwell plates are sipped into a capillary or capillaries attached to the chip and drawn into the channels of the chip. There they are mixed with components of interest and a processing and result detection steps are performed.

Whether conventional fluid handling or microfluidic approaches (or both) are used, the aliquotting and/or dilution or other fluid handling events can be performed to achieve particular results. For example, a sample can be diluted equally in each aliquot, or, alternately, the aliquots can be differentially diluted (e.g., a dilution series can be made). The aliquots themselves are of a volume that is appropriate to the fluid handling approach being used by the system, e.g., on the order of a few microliters for microtiter plates to 100 nL, 10 nL or even 1 nL or less for microfluidic approaches. This dilution approach can be one mechanism for optimizing a reaction for a microscale system, e.g., to provide appropriate concentrations of template or other materials to provide robust amplification without clogging a microscale reaction chamber with amplicon produced by the system.

Downstream Processing of Nucleic Acid Amplicons

Host cells can be transduced with nucleic acids produced according to the methods herein (or, e.g., cDNA copies thereof), e.g., cloned into vectors, for production of nucleic acids and expression of encoded molecules. In addition to Berger, Sambrook and Ausubel, a variety of references, including, e.g., Freshney (1994) Culture of Animal Cells, a Manual of Basic Technique, third edition, Wiley-Liss, New York and the references cited therein, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fl. provide additional details on cell culture, cloning and expression of nucleic acids in cells.

Transcription/Translation

In one aspect, the invention optionally includes in vitro translation of one or more RNAs produced in the amplification procedures noted above. Advantageously, this translation can be performed in a microscale system that the RNA amplification protocol was performed in, resulting in a system that produces proteins encoded by the RNAs. These proteins can be detected by standard protein detection methods, e.g., by assaying for a function of the protein or detecting binding of an antibody, aptamer or other detectable molecule that specifically binds the translated protein.

Either of at least two different approaches can be used. First, the RNA of interest can be cloned into an appropriate expression vector (typically by reverse transcribing the RNA into DNA and then cloning the DNA into the vector) as noted above. The protein is then produced by expressing the protein in the expression vector by convention techniques. Many different cloning methods are set forth in Ausubel, Sambrook, Alberts and Lodish, all supra. Additional details can be found in Berger and Kimmel, Guide to Molecular Cloning Techniques, Methods in Enzymology volume 152 Academic Press, Inc., San Diego, Calif. (Berger). Advantageously, one or more of the steps of the cloning procedure can be performed in a microscale device, e.g., by flowing appropriate templates, cloning vectors, enzymes, buffers, cells or the like into contact and incubating the components according to the relevant cloning protocol.

In a second, and generally preferable approach, the RNA is translated into a protein directly in vitro. If the complementary strand is more desirably expressed, the RNA is reverse translated, and the correct strand of DNA replicated into RNA, which is then translated in vitro. Common in vitro transcription and/or translation reagents include reticulocyte lysates (e.g., rabbit reticulocyte lysates) wheat germ in vitro translation (IVT) mixtures, E coli lysates, canine microsome systems, HeLa nuclear extracts, the “in vitro transcription component,” (see, e.g., Promega technical bulletin 123), SP6 polymerase, T3 polymerase, T7 RNA polymerase (e.g., Promega # TM045), the “coupled in vitro transcription/translation system” (Progen Single Tube Protein System 3) and many others. Many of translation systems are described, e.g., in Ausubel, supra, as well as in the references below, and many transcription/translation systems are commercially available.

Methods of processing (replicating, transcribing and/or translating) nucleic acids are provided herein, e.g., as specially adapted for microfluidic systems. One or more in vitro amplification, transcription or translation product produced by the methods is optionally detected. The reaction mixtures and systems comprising these mixtures are also a feature of the invention.

Generally, cell-free transcription/translation systems can be employed to produce polypeptides from solid or liquid phase mixtures of RNAs or DNAs (e.g., cDNAs) as provided by the present invention. Several transcription/translation systems are commercially available and can be adapted to the present invention by the appropriate incorporation of transcription and or translation reagents to source wells in the microscale systems of the invention. A general guide to in vitro transcription and translation protocols is found in Tymms (1995) In vitro Transcription and Translation Protocols: Methods in Molecular Biology Volume 37, Garland Publishing, NY. Any of the reagents used in these systems can be flowed or otherwise directed into contact with amplified or transcribed nucleic acids of the invention. Typically, in the present invention, in vitro transcription and/or translation reagents are mixed in a microchamber and then processed in a microscale system, or removed from the microscale system and analyzed according to standard methods.

Several in vitro transcription and translation systems are well known and described in Tymms (1995), id. For example, an untreated reticulocyte lysate is commonly isolated from rabbits after treatment of the rabbits with acetylphenylhydrazine as a cell-free in vitro translation system. Similarly, coupled transcription/translation systems often utilize an E. coli S30 extract. See also, the Ambion 1999 Product Catalogue from Ambion, Inc (Austin Tex.).

A variety of in vitro transcription and translation reagents are commercially available, including the PROTEINscript-PRO™ kit (for coupled transcription/translation) the wheat germ IVT kit, the untreated reticulocyte lysate kit (each from Ambion, Inc (Austin Tex.)), the HeLa Nuclear Extract in vitro Transcription system, the TnT Quick coupled Transcription/translation systems (both from Promega, see, e.g., Technical bulletin No. 123 and Technical Manual No. 045), and the single tube protein system 3 from Progen. Each of these available systems (as well as many other available systems) have certain advantages, detailed by the product manufacturer.

In addition, the art provides considerable detail regarding the relative activities of different in vitro transcription translation systems, for example as set forth in Tymms, id.; Jermutus et al. (1999) “Comparison of E. coli and rabbit reticulocyte ribosome display systems” FEBS Lett. 450(1-2):105-10 and the references therein; Jermutus et al. (1998) “Recent advances in producing and selecting functional proteins by using cell-free translation” Curr. Opin. Biotechnol. 9(5):534-48 and the references therein; Hanes et al. (1988) “Ribosome Display Efficiently Selects and Evolves High-Affinity Antibodies in vitro from Immune Libraries” PNAS 95:14130-14135 and the references therein; and Hanes and Pluckthun (1997) “In vitro Selection and Evolution of Functional Proteins by Using Ribosome Display.” Biochemistry 94:4937-4942 and the references therein.

For example, an untreated rabbit reticulocyte lysate is suitable for initiation and translation assays where the prior removal of endogenous globin mRNA is not necessary. The untreated lysate translates exogenous mRNA, but also competes with endogenous mRNA for limiting translational machinery.

Similarly, The PROTEINscript-PRO™ kit from Ambion is designed for coupled in vitro transcription and translation using an E. coli S30 extract. In contrast to eukaryotic systems, where the transcription and translation processes are separated in time and space, prokaryotic systems are coupled, as both processes occur simultaneously. During transcription, the nascent 5′-end of the mRNA becomes available for ribosome binding, allowing transcription and translation to proceed at the same time. This early binding of ribosomes to the mRNA maintains transcript stability and promotes efficient translation. Coupled transcription: translation using the PROTEINscript-PRO Kit is based on this E. coli model.

The Wheat Germ IVT™ Kit from Ambion, or other similar systems, is/are a convenient alternative, e.g., when the use of a rabbit reticulocyte lysate is not appropriate for in vitro protein synthesis. The Wheat Germ IVT™ Kit can be used, e.g., when the desired translation product comigrates with globin (approx. 12-15 kDa), when translating mRNAs coding for regulatory factors (such as transcription factors or DNA binding proteins) which may already be present at high levels in mammalian reticulocytes, but not plant extracts, or when an mRNA will not translate for unknown reasons and a second translation system is to be tested.

The TNT® Quick Coupled Transcription/Translation Systems (Promega) are single-tube, coupled transcription/translation reactions for eukaryotic in vitro translation. The TNT® Quick Coupled Transcription/Translation System combines RNA Polymerase, nucleotides, salts and Recombinant RNasin® Ribonuclease Inhibitor with the reticulocyte lysate to form a single TNT® Quick Master Mix. The TNT® Quick Coupled Transcription/Translation System is available in two configurations for transcription and translation of genes cloned downstream from either the T7 or SP6 RNA polymerase promoters. Included with the TNT® Quick System is a luciferase-encoding control plasmid and Luciferase Assay Reagent, which can be used in a non-radioactive assay for rapid (<30 seconds) detection of functionally active luciferase protein.

Many other systems are well known, well characterized and set forth in the references noted herein, as well as in other references known to one of skill. It will also be appreciated that one of skill can produce transcription/translation systems similar to those which are commercially available from available materials, e.g., as taught in the references noted above.

Once expressed, proteins can be purified and/or detected, either partially or substantially to homogeneity, according to standard procedures known to and used by those of skill in the art. These procedures can be adapted to microscale systems, but do not have to be, as one may simply collect proteins or RNA (or DNA) products and from output wells of a microscale system and analyze the products according to any available method. These purification methods include ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in completing configuration of mature proteins. High performance liquid chromatography (HPLC) can be employed in purification steps where high purity is desired. Once purified, partially or to homogeneity, as desired, the polypeptides may be used (e.g., as assay components, therapeutic reagents or as immunogens for antibody production).

In addition to the references noted supra, a variety of protein manipulation methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, N.Y.; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

As noted, those of skill in the art will recognize that after synthesis, expression and/or purification, proteins can possess a conformation substantially different from the native conformations of the relevant parental polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCI. In general, it is occasionally desirable to denature and reduce expressed polypeptides and then to cause the polypeptides to re-fold into the preferred conformation. For example, guanidine, urea, DTT, DTE, and/or a chaperonin can be added incubated with a transcription product of interest. Methods of reducing, denaturing and renaturing proteins are well known to those of skill in the art (see, the references above, and Debinski, et al. (1993) J. Biol. Chem., 268: 14065-14070; Kreitman and Pastan (1993) Bioconjug. Chem. 4: 581-585; and Buchner, et al., (1992) Anal. Biochem., 205: 263-270). Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

Various systems are also available for simultaneous synthesis and folding of complex proteins. For example, the control of redox potential, the use of helper proteins (from both bacterial and eukaryotic systems) and the like can be used to provide for improved cell free translation.

Use of the Nucleic Acids of the Invention as Inhibitors: Antisense and RNAi

In addition to serving as substrates for translation, or as reagents for binding nucleic acids, RNAs made according to the present invention can also be used as inhibitors of gene expression, e.g., in any therapeutic or diagnostic assay that relies upon suppressing gene expression. For example, the use of RNAi for inhibiting gene expression in a number of cell types (including, e.g., mammalian cells) and organisms is well described in the literature, as are methods for determining appropriate interfering RNA(s) to target a desired gene and for generating such interfering RNAs. For example, RNA interference is described e.g., in U.S. patent application publications 20020173478, 20020162126, and 20020182223 and in Hannon (2002) “RNA interference” Nature 418 (6894):244-51; Ueda (2001) “RNAi: a new technology in the post-genomic sequencing era” J Neurogenet. 15(3-4):193-204; Ullu et al (2002) “RNA interference: advances and questions” Philos Trans R Soc Lond B Biol Sci. 357(1417):65-70; and Schmid et al. (2002) “Combinatorial RNAi: a method for evaluating the functions of gene families in Drosophila” Trends Neurosci. 25 (2):71-4. A kit for producing interfering RNAs is commercially available, e.g., from Ambion, Inc. (www.ambion.com, the Silencer™ siRNA construction kit); kits for labeling such RNAs are available from the same source.

Similarly, an antisense nucleic acid can be produced by the methods of the invention, e.g., for any gene whose coding sequence is known or can be determined. Antisense nucleic acids and their use are described, e.g., in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Uhlmann and A. Pepan, Chem. Rev. 90 (1990) 543; P. D. Cook, Anti-Cancer Drug Design 6 (1991) 585; J. Goodchild, Bioconjugate Chem. 1 (1990) 165; S. L. Beaucage and R. P. Iyer, Tetrahedron 49 (1993) 6123; and F. Eckstein, Ed., “Oligonucleotides and Analogues—A Practical Approach”, IRL Press (1991).

The nucleic acids made according to the present invention can also be catalytic nucleic acids, such as catalytic RNAs (“ribozymes”), which can also be used to inhibit nucleic acid expression. A variety of ribozymes that can be made according to the present invention are known. For example, see, Castanotto et al. (1994) Adv. in Pharmacology 25: 289-317; Scott (1999) “Biophysical and biochemical investigations of RNA catalysis in the hammerhead ribozyme” Quarterly Reviews of Biophysics 32, 3, 241-284; Marschall et al. (1994) “Inhibition of gene expression with ribozymes” Cell Mol Neurobiol. 14(5):523-38; Hampel et al. (1990) Nucl. Acids Res. 18: 299-304; Hampel et al. (1990) European Patent Publication No. 0 360 257; and U.S. Pat. No. 5,254,678.

Systems for Performing Nucleic Acid Amplification

A number of systems, including microscale systems, exist for performing nucleic acid amplification. Theses systems can be adapted to the present invention by optimizing reaction conditions in the systems to provide for continuous flow amplification of nucleic acids such as RNAs, e.g., in a relevant amplification format (e.g., by providing Van Gelder-Eberwine reagents to the reaction chambers of microscale systems). Such systems include those adapted to performing PCR and other amplification reactions, as well as methods for detecting and analyzing amplified nucleic acids in or on the devices. Details regarding such technology is found in the technical and patent literature, e.g., Kopp et al. (1998) “Chemical Amplification: Continuous Flow PCR on a Chip” Science, 280 (5366):1046; U.S. Pat. No. 6,444,461 to Knapp, et al. (Sep. 3, 2002) MICROFLUIDIC DEVICES AND METHODS FOR SEPARATION; U.S. Pat. No. 6,406,893 to Knapp, et al. (Jun. 18, 2002) MICROFLUIDIC METHODS FOR NON-THERMAL NUCLEIC ACID MANIPULATIONS; U.S. Pat. No. 6,391,622 to Knapp, et al. (May 21, 2002) CLOSED-LOOP BIOCHEMICAL ANALYZERS; U.S. Pat. No. 6,303,343 to Kopf-Sill (Oct. 16, 2001) INEFFICIENT FAST PCR; U.S. Pat. No. 6,171,850 to Nagle, et al. (Jan. 9, 2001) INTEGRATED DEVICES AND SYSTEMS FOR PERFORMING TEMPERATURE CONTROLLED REACTIONS AND ANALYSES; U.S. Pat. No. 5,939,291 to Loewy, et al. (Aug. 17, 1999) MICROFLUIDIC METHOD FOR NUCLEIC ACID AMPLIFICATION; U.S. Pat. No. 5,955,029 to Wilding, et al. (Sep. 21, 1999) MESOSCALE POLYNUCLEOTIDE AMPLIFICATION DEVICE AND METHOD; U.S. Pat. No. 5,965,410 to Chow, et al. (Oct. 12, 1999) ELECTRICAL CURRENT FOR CONTROLLING FLUID PARAMETERS IN MICROCHANNELS; Service (1998) “Microchips Arrays Put DNA on the Spot” Science 282:396-399), Zhang et al. (1999) “Automated and Integrated System for High-Throughput DNA Genotyping Directly from Blood” Anal. Chem. 71:1138-1145 and many others. These systems can be adapted to the present invention by configuring the systems to perform an amplification reaction, or other appropriate reaction (e.g., translation) in an optimized manner.

For example, U.S. Pat. No. 6,391,622 to Knapp, et al. (May 21, 2002) CLOSED-LOOP BIOCHEMICAL ANALYZERS and the references cited therein describe systems comprising microfluidic elements that can access reagent storage systems and that can perform PCR or other amplification reactions by any of a variety of methods in the microfluidic system. For example, the microfluidic system can have one or more capillaries extending outwards from the body structure of the microfluidic system for drawing materials into the body structure. Within the body structure are microfluidic cavities (channels, chambers, or the like having at least one dimension smaller than about 500 microns, and, typically smaller than about 100 microns) in which the amplification reactions are performed. The capillaries that extend out from the body structure can access standard reagent storage elements (microtiter plates, or the like) by drawing fluid into the capillary, e.g., due to application of a vacuum or electroosmotic force. Similarly, the capillaries can access dried reagent libraries on substrates (e.g., LIBRARYCARD™ reagent library made by Caliper Life Sciences, Inc.) by expelling fluid to rehydrate library members and then by drawing the rehydration fluid back into the capillary. In either case, molecular beacons or TaqMan™ probes can be incorporated into the relevant amplification reaction and detected in the microfluidic device to provide for real time PCR detection. Alternately, PCR amplicons can be detected by conventional methods, such as hybridization to a labeled probe, e.g., prior to or following a separation operation that separates unhybridized probe from hybridized probe. For example, an electrophoretic separation can be performed in a channel of the microscale device.

Any of these systems is adapted to the present invention, e.g., by including reagents for RNA amplification (e.g., any of those noted herein, including, e.g., those used in the Van Gelder-Eberwine reaction and configuring the system to optimize RNA or other amplicon production by the system. For example, the system RNA amplification reagents can include a solid support, the template nucleic acid, a DNA template, a poly(dT) oligonucleotide with an RNA polymerase promoter sequence, a cell, a cell extract, a reverse transcriptase, an rNTP, a dNTP, Mg++, and/or a buffer. The system can include the sample to be amplified, e.g., a plurality of cDNAs that encode total polyA mRNA from a biological sample, or can simply include the relevant RNAs, if the system is configured to perform reverse transcription. Similarly, the sample can include 1 or more cells (e.g., 10 or more, 100 or more, 1,000 or more, etc.).

The reaction parameter can be any of those noted herein, e.g., a rate of flow in the reaction chamber, a temperature in the chamber, a concentration of one or more of the RNA amplification reagents in the chamber, inhibiting or enhancing DNA transcription in the amplification chamber, a channel size leading into or out of the chamber, a size of the chamber, a bead diameter of a bead bound to one or more additional RNA amplification reagent, total porosity of a bead bed bound to one or more additional RNA amplification reagent, a percent of fluid that diffuses in and out of a bead bed bound to one or more additional RNA amplification reagent as the fluid flows through and along the bead bed, residence time of (and distance traveled by) the reaction substrates or products through the bead bed, etc. For example, in one embodiment, reactants are flowed through a bead bed (which can be, e.g., in a channel, chamber or well) in a direction transverse (orthogonal) to flow of products out of the bead bed. For example, reactants can be flowed into the bead bed along a long dimension of the bead bed (reactants generally can generally flow relatively freely through a bead bed), while products are flowed across a short dimension of the bead bed (products can be more resistant to flow through the bead bed, and yields can be improved by configuring the flow path of products for reduced flow). Alternately, reagents and products can both be flowed through the short dimension of the bead bed, minimizing trapping by the bead bed. In either embodiment, the beads themselves are optionally flowed in a direction transverse (orthogonal) to the flow of the reactants and/or products.

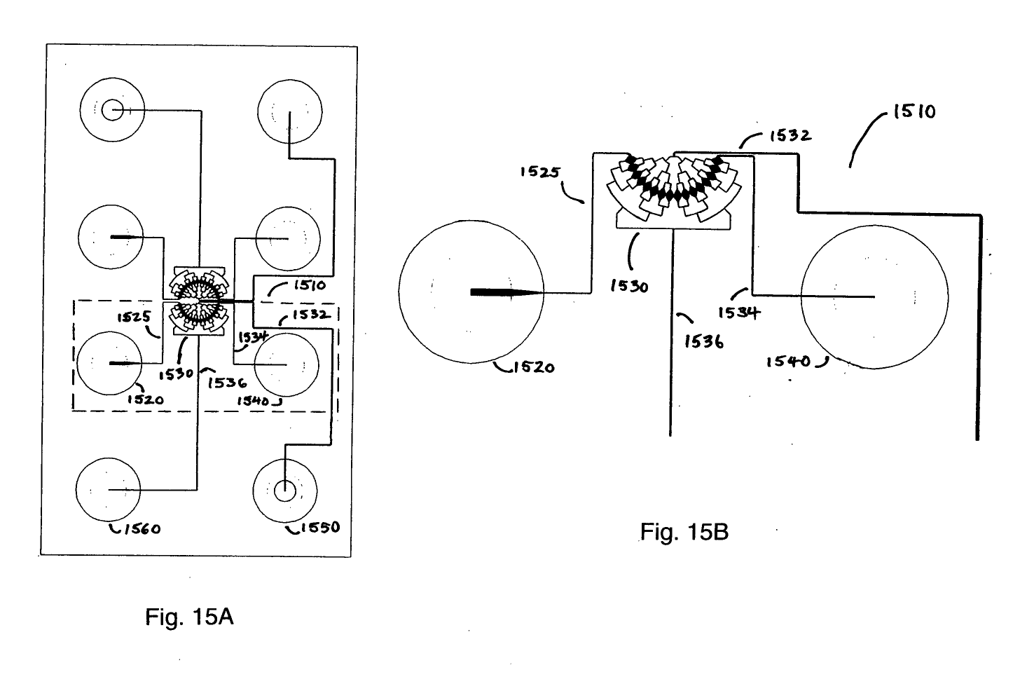

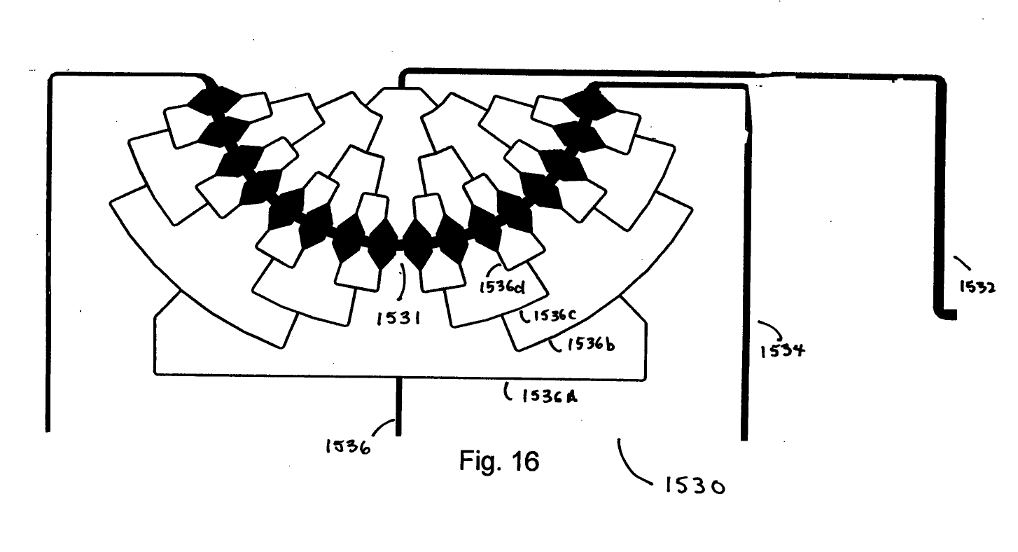

Two such embodiments are illustrated in FIGS. 15-17. The first embodiment is shown in FIGS. 15 and 16. FIG. 15A shows the pattern of channels and chambers on a microfluidic device 1500 containing two microfluidic networks, each network comprising a reaction chamber 1530, channels 1525,1532,1534,1536 for moving materials into and out of the reaction chamber 1530, and reservoirs 1520,1540,1550,156 in fluid communication with the channels. The two networks are mirror images of each other, and are symmetrical about a horizontal line bisecting the top and bottom halves of the microfluidic device 1530. A portion 1510 of the microfluidic network in the bottom half of the microfluidic device 1500 is shown in more detail in FIG. 15B. The reaction chamber 1530 that same microfluidic network is shown in even more detail in FIG. 16. In the embodiment of FIGS. 15 and 16, beads are flowed from reservoir 1520 through channel 1525 to form a bead bed 1531 in the reaction chamber 1530. The beads flow through the bead bed 1531 into channel 1534, which leads to a waste storage reservoir 1540. Reagents stored in reservoir 1560 are transported into the bead bed 1531 in the reaction chamber 1530 via channel 1536. In the vicinity of the bead bed 1531, channel 1536 is divided and redivided into a series of channels, such as channels 1536a, 1536b, 1536c, 1536d, so that the flow of reactants into bead bed 1531 is distributed across the length of the bed. The final set of channels entering the side of the bead bed 1531, such as channel 1536d, also increase in width as they enter the bead bed to further the distribution of reactants in the bed. Widening the distribution of reactants increases the amount of bead surface area contacted by the reactants. The reactants flow across the width, which is the shorter dimension (compared to the length), of the bead bed 1531. After crossing the width of the bead bed 1531, the flow from channel 1536 will comprise reaction products as well as any unreacted reagents. When the flow comprising the reaction products exits the bead bed 1531 it enters a series of channels that coalesce into channel 1532. The reaction products of interest may be detected as they flow through channel 1532, or after they are collected in reservoir 1550. leads to reservoir that that the incoming reactants and those two streams The flow of reactants and products across the width of the bead bed 1531 is transverse to the flow of beads along the length of the bead bed. The channel forming bead bed 1531 is deeper than the channels (e.g. 1536d) transporting reactants and products across the bed so that the flow of beads is constrained to the deeper bead bed channel.

As previously discussed, one feature of the invention is the optimization of reaction parameters such as flow rates, reaction temperatures, reagent concentrations, channel geometry, reaction chamber geometry, and bead diameter, for the performance of RNA amplification in microscale systems. For a microscale system comprising the microfluidic device shown in FIGS. 15 and 16, for example, the system could be optimized to accommodate glass beads with a diameter of 6.5-10 μm. To prevent flow of the beads from the bead bed 1531 into the channels entering and exiting the bed along its length, those channels must be shallow enough to prevent the entry of the beads. So, for example, channels 1532 and 1536, and the series of channels connecting channels 1532 and 1536 to the bead bed 1531, could be fabricated with a depth of 6 μm or less. The bead bed could be fabricated to a depth wide enough to accommodate the desired flow of beads. So, for example, the bead bed could be fabricated with a depth of approximately 25 μm. Methods of flowing beads through microfluidic channels are known in art, and are described in U.S. Pat. No. 6,632,655 entitled “Manipulation of microparticles in microfluidic systems.” Methods of fabricating microfluidic devices comprising channels of different depths are also known in the art, and are described in U.S. Pat. No. 6,569,607 entitled “Multi depth substrate fabrication processes.” Once the depths of the various channels and chambers in the microfluidic device 1500 have been determined, the widths of the channels can be chosen to as to provide the desired fluid flow rates when the available driving forces are delivered to the fluid. For example, if fluid flow through channel 1536, across the width of the bead bed 1531, and finally through channel 1536 is to be provided by a pressure source capable of providing +/−5 psig, and the desired flow rate through channels 1536 and 1532 is approximately 6 μl/hr, then making the width of channels 1536 and 1532 approximately 70-100 μm, and making the width of the series of channels connecting channels 1536 and 1532 to the bead bed approximately 15 μm, applying a pressure of 5 psig to reservoir 1560 while applying −5 psig to reservoir 1550 will provide the desired flow rate. As will be understood by those skilled in the art, similar flow rates can be achieved through different combinations of driving forces, fluid properties such as viscosity, and channel dimensions. It will also be understood by those in the art that the residence time of the reactants flowing through the bead bed 1531 will be determined by both the flow rate of the reactants, e.g. 6 μl/hr, and the volume of the bead bed. For example, a sufficient residence time to carry out RNA amplification in a bead bed 1531 with a depth of 25 μm could be provided by fabricating the bed to have a width of between 70 and 100 μm. Note that there should be little flow towards reservoirs 1520 and 1540 if those reservoirs are left open to the atmosphere because of the negative pressure (with respect to atmospheric) being applied to reservoir 1550.

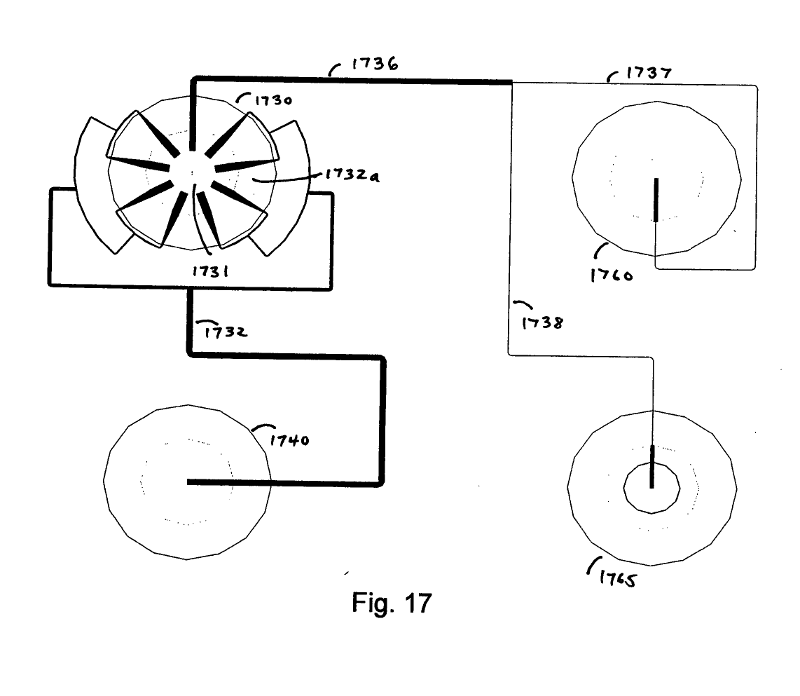

FIG. 17 provides a schematic of a device having similar features to the device in FIGS. 15 and 16, with an alternate star patterned geometry. In the embodiment of FIG. 17, reagents from two different reagent reservoirs 1760,1765 flow through channels 1737 and 1738 respectively toward reaction chamber 1730. Before reaching the reaction chamber 1730, the channels 1737,1738 carrying the reagents converge into a single reagent channel 1736. The flow of reagents from channel 1736 enters the bead bed 1731 in the reaction chamber. The flow containing reaction products, as well and any unreacted reagents, exits the bead bed 1731 through a series of channels, such as channel 1732a, that radiate outward from the circumference of bead bed 1731. The configuration of channels entering and leaving the bead bed 1731 results in a flow pattern in which the reagents flow into the bead bed in a direction transverse (although not orthogonal) to the directions in which products flow out of the bead bed. The channels exiting the bead bed 1731 coalesce into channel 1732, which directs reaction products from the reaction chamber 1730 toward reservoir 1740. The reaction products of interest can be detected while flowing through channel 1732, or once they are collected in reservoir 1740. Channel dimensions, pressure driving forces, and flow rates similar to those previously described with respect to the embodiment of FIGS. 15 and 16 are compatible with the embodiment of FIG. 17.

In another similar embodiment, one or more additional RNA amplification reagent is optionally contained within a bead bed that fills a deep portion of a microscale channel, that has a lateral step up in depth. The system is configured so that one or more amplification reagents and amplicons are flowed along a side of the bead bed and can diffuse laterally in and out of the bead bed. The system can be so configured by placing appropriate amplification reagents in reagent sources coupled to or integral with the system (e.g., wells on a microscale device) and providing the system with instructions (e.g., through user-configured system software) to flow reagents in the appropriate optimized manner into the reaction chamber (e.g., comprising the bead bed)

Detecting the Amplified Nucleic Acids

Any available method for detecting amplified nucleic acids can be used in the present invention. Common approaches include real time amplification detection with molecular beacons or TaqMan™ probes, detection of intercalating dyes, detection of labels incorporated into the amplification probes or the amplified nucleic acids themselves, e.g., following electrophoretic separation of the amplification products from unincorporated label), hybridization based assays (e.g., array based assays) and/or detection of secondary reagents that bind to the nucleic acids. Details on these general approaches is found in the references cited herein, e.g., Sambrook, Ausubel, and the references in the sections herein related to real time PCR detection. Additional labeling strategies for labeling nucleic acids and corresponding detection strategies can be found, e.g., in Haugland (2003) Handbook of Fluorescent Probes and Research Chemicals Ninth Edition by Molecular Probes, Inc. (Eugene Oreg.) (Also available on CD ROM). Nucleic Acid Specific incorporation of Florescent Dyes

Detecting Amplicons in a Solution Phase Assay

Amplification products can be detected in a solution phase, eliminating any need for size/charge separation and/or hybridization or sequencing (although these approaches can be used, if desired, to provide additional information of what sequences are being detected), for a number of applications. For example, the amount of a double-stranded DNA amplicon can be determined by monitoring double-strand DNA specific dye incorporation by the amplicon. Similarly, direct detection of RNA products can be practiced by monitoring dye-specific incorporation of RNA Dyes.

Detection with Specific Dyes

In one aspect of the invention, detecting and quantifying RNA is useful, for any of a wide variety of molecular biology procedures, including those herein. These can include measuring yields of in vitro transcribed RNA and measuring RNA concentrations after amplification and/or before performing Northern blot analysis, S1 nuclease assays, RNase protection assays, cDNA library preparation, reverse transcription PCR, differential display PCR, or other assays on the amplified RNA. A number of RNA specific dyes are available, such as RiboGreen®, which is one of the more commonly used dyes for solution phase RNA detection. This dye is an example of an ultrasensitive fluorescent nucleic acid stain for quantifying RNA in solution and is available from Molecular Probes (catalogue No. R-11491 and R-11490). RiboGreen RNA can quantify as little as 1 ng/mL RNA with a standard spectrofluorometer or filter fluorometer, using fluorescein excitation and emission wavelengths. The excitation maximum for RiboGreen reagent bound to RNA is ˜500 nm and the emission maximum is ˜525 nm. The linear range for RiboGreen quantification extends over three orders of magnitude in RNA concentration—from 1 ng/mL to 1 μg/mL RNA.

Similarly, the PicoGreen dye is a double-stranded DNA-specific dye (available, e.g., from Molecular Probes) that can be used to monitor and quantify double stranded DNA amplicons. Similarly, an OliGreen single stranded DNA-specific reagent can be used to monitor and quantify ssDNA amplification products. RiboGreen is an RNA quantification reagent that can be used to monitor formation of RNA. See, e.g., Haugland (2003). For example, Molecular Probes Chapter 8 provides details regarding quantification of DNA in solution.

The PicoGreen reagent (e.g., Molecular Probes Nos. P-758 1, P-11495) and Kit (Molecular Probes Nos. P-7589, P-11496) accurately quantify as little as 25 pg/mL of double-stranded DNA (dsDNA) in a fluorometer or 250 pg/mL (typically 50 pg in a 200 μL volume) in a fluorescence microplate reader. The PicoGreen assay is greater than 10,000 times more sensitive than conventional UV absorbance measurements at 260 nm (an A260 of 0.1 corresponds to a 5 μg/mL dsDNA solution). Although the PicoGreen reagent is not actually specific for dsDNA, it shows a >1000-fold fluorescence enhancement upon binding to dsDNA, and less fluorescence enhancement upon binding to single-stranded DNA (ssDNA) or RNA, making it possible to quantify dsDNA in the presence of ssDNA, RNA, proteins or other materials. Thus, the PicoGreen reagent allows direct quantification of, e.g., PCR amplicons without purification from the reaction mixture and makes it possible to detect low levels of DNA contamination in recombinant protein products.

The protocol for the PicoGreen assay is amenable to high-throughput screening in the systems herein—the dye is added to the product sample (e.g., in a microchannel) and incubated for about five minutes, and then the fluorescence is measured. In addition, the fluorescence signal from binding of the PicoGreen reagent to dsDNA is linear over at least four orders of magnitude with a single dye concentration. Linearity is maintained in the presence of several compounds commonly found in nucleic acid preparations, including salts, urea, ethanol, chloroform, detergents, proteins and agarose.

For detecting ssDNA amplicons in solution, the OliGreen ssDNA quantification reagent from Molecular Probes (No. O-7582) and/or (No. O-11492) can be used). The OliGreen ssDNA quantification reagent enables quantification of as little as 100 pg/mL of ssDNA using a standard fluorescence reader. Thus, quantification with the OliGreen reagent is about 10,000 times more sensitive than quantification with UV absorbance methods and at least 500 times more sensitive (and far faster, with a greater throughput) than detecting oligonucleotides on electrophoretic gels stained with ethidium bromide.

The solution phase OliGreen ssDNA quantitation reagent does exhibit fluorescence enhancement when bound to dsDNA and RNA. Like the PicoGreen assay, the linear detection range of the OliGreen assay in a standard fluorometer extends over four orders of magnitude—from 100 pg/mL to 1 μg/mL—with a single dye concentration. The linearity of the OliGreen assay is also maintained in the presence of several compounds commonly found to contaminate nucleic acid preparations, including salts, urea, ethanol, chloroform, detergents, proteins, ATP and agarose (see, e.g., the OliGreen product information sheet from Molecular Probes); however, many of these compounds do affect signal intensity, so standard curves are typically generated using solutions that closely mimic those of the samples. The OliGreen reagent shows a large fluorescence enhancement when bound to poly(dT) but only a relatively small fluorescence enhancement when bound to poly(dG) and little signal with poly(dA) and poly(dC). Thus, it is helpful to use an oligonucleotide with similar base composition when generating a standard curve for concentration dependence. The OliGreen ssDNA quantification reagent can be used for quantitation of any single stranded DNA amplicon.

Other dyes such as the Cyanine Dyes and Phenanthridine Dyes can also be used for nucleic acid quantification in solution and are, therefore, adaptable to use in the present invention. See, Molecular Probes, Supra, for a discussion of these and many other nucleic acid staining and quantification dyes.

Solution Phase Detection—Molecular Beacons or TaqMan

In one aspect, molecular beacons or TaqMan oligonucleotide based detection methods are used for real time detection of an amplified nucleic acid (RNA or DNA) of interest.

Molecular beacons (MBs) are oligonucleotides designed for real time detection and quantification of target nucleic acids (e.g., target DNAs). The 5′ and 3′ termini of MBs collectively comprise a pair of moieties which confers the detectable properties of the MB. One of the termini is attached to a fluorophore and the other is attached to a quencher molecule capable of quenching a fluorescent emission of the fluorophore. For example, one example fluorophore-quencher pair can use a fluorophore such as EDANS or fluorescein, e.g., on the 5′-end and a quencher such as Dabcyl, e.g., on the 3′-end. When the MB is present free in solution, i.e., not hybridized to a second nucleic acid, the stem of the MB is stabilized by complementary base pairing. This self-complementary pairing results in a “hairpin loop” structure for the MB in which the fluorophore and the quenching moieties are proximal to one another. In this confirmation, the fluorescent moiety is quenched by the fluorophore. The loop of the molecular beacon is complementary to a sequence to be detected in the target nucleic acid, such that hybridization of the loop to its complementary sequence in the target forces disassociation of the stem, thereby distancing the fluorophore and quencher from each other. This results in unquenching of the fluorophore, causing an increase in fluorescence of the MB.

Details regarding standard methods of making and using MBs are well established in the literature and MBs are available from a number of commercial reagent sources. Further details regarding methods of MB manufacture and use are found, e.g., in Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11: 187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

MB components (e.g., oligos, including those labeled with fluorophores or quenchers) can be synthesized using conventional methods. For example, oligos or peptide nucleic acids (PNAS) can be synthesized on commercially available automated oligonucleotide/PNA synthesis machines using standard methods. Labels can be attached to the oligos or PNAs either during automated synthesis or by post-synthetic reactions which have been described before see, e.g., Tyagi and Kramer (1996) “Molecular beacons: probes that fluoresce upon hybridization” Nature Biotechnology 14:303-308 and U.S. Pat. No. 6,037,130 to Tyagi et al (Mar. 14, 2000), entitled “Wavelength-shifting probes and primers and their use in assays and kits.” and U.S. Pat. No. 5,925,517 (Jul. 20, 1999) to Tyagi et al. entitled “Detectably labeled dual conformation oligonucleotide probes, assays and kits.” Additional details on synthesis of functionalized oligos can be found in Nelson, et al. (1989) “Bifunctional Oligonucleotide Probes Synthesized Using A Novel CPG Support Are Able To Detect Single Base Pair Mutations” Nucleic Acids Research 17:7187-7194. Labels/quenchers can be introduced to the oligonucleotides or PNAs, e.g., by using a controlled-pore glass column to introduce, e.g., the quencher (e.g., a 4-dimethylaminoazobenzene-4′-sulfonyl moiety (DABSYL). For example, the quencher can be added at the 3′ end of oligonucleotides during automated synthesis; a succinimidyl ester of 4-(4′-dimethylaminophenylazo)benzoic acid (DABCYL) can be used when the site of attachment is a primary amino group; and 4-dimethylaminophenylazophenyl-4′-maleimide (DABMI) can be used when the site of attachment is a sulphydryl group. Similarly, fluorescein can be introduced in the oligos, either using a fluorescein phosphoramadite that replaces a nucleoside with fluorescein, or by using a fluorescein dT phosphoramadite that introduces a fluorescein moiety at a thymidine ring via a spacer. To link a fluorescein moiety to a terminal location, iodoacetoamidofluorescein can be coupled to a sulphydryl group. Tetrachlorofluorescein (TET) can be introduced during automated synthesis using a 5′-tetrachloro-fluorescein phosphoramadite. Other reactive fluorophore derivatives and their respective sites of attachment include the succinimidyl ester of 5-carboxyrhodamine-6G (RHD) coupled to an amino group; an iodoacetamide of tetramethylrhodamine coupled to a sulphydryl group; an isothiocyanate of tetramethylrhodamine coupled to an amino group; or a sulfonylchloride of Texas red coupled to a sulphydryl group. During the synthesis of these labeled components, conjugated oligonucleotides or PNAs can be purified, if desired, e.g., by high pressure liquid chromatography or other methods.

A variety of commercial suppliers produce standard and custom molecular beacons, including Cruachem (cruachem.com), Oswel Research Products Ltd. (UK; oswel.com), Research Genetics (a division of Invitrogen, Huntsville Ala. (resgen.com)), the Midland Certified Reagent Company (Midland, Tex. mcrc.com) and Gorilla Genomics, LLC (Alameda, Calif.). A variety of kits which utilize molecular beacons are also commercially available, such as the Sentinel™ Molecular Beacon Allelic Discrimination Kits from Stratagene (La Jolla, Calif.) and various kits from Eurogentec SA (Belgium, eurogentec.com) and Isogen Bioscience BV (The Netherlands, isogen.com).

In one embodiment, a real time PCR assay system such as the “TaqMan” system is used for detecting amplified nucleic acids. TaqMan operates by using the endogenous endonuclease activity of certain polymerases to cleave a quencher or label free from an oligonucleotide that comprises the quencher and label, resulting in unquenching of the label. The polymerase only cleaves the quencher or label upon initiation of replication, i.e., when the oligonucleotide is bound to the template and the polymerase extends the primer. Thus, an appropriately labeled oligonucleotide and polymerase comprising the appropriate nuclease activity can be used to detect a nucleic acid of interest. Real time PCR product analysis by, e.g., FRET or TaqMan (and related real time reverse-transcription PCR) provides a well-known technique for real time PCR monitoring that has been used in a variety of contexts (see, Laurendeau et al. (1999) “TaqMan PCR-based gene dosage assay for predictive testing in individuals from a cancer family with INK4 locus haploinsufficiency” Clin Chem 45(7):982-6; Laurendeau et al. (1999) “Quantitation of MYC gene expression in sporadic breast tumors with a real-time reverse transcription-PCR assay” Clin Chem 59(12):2759-65; and Kreuzer et al. (1999) “LightCycler technology for the quantitation of bcr/abl fusion transcripts” Cancer Research 59(13):3171-4.

Probe Synthesis Methods

In general, synthetic methods for making oligonucleotides, including probes, molecular beacons, PNAs, LNAs (locked nucleic acids), etc., which can be used as reagents for the detection of nucleic acid amplicons made according to the methods herein, are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. Similarly, PNAs can be custom ordered from any of a variety of sources, such as PeptidoGenic (pkim@ccnet.com), HTI Bio-products, inc. (www.htibio.com), BMA Biomedicals Ltd (U.K.), Bio-Synthesis, Inc., and many others.

Size/Charge Based Detection Methods

In one additional preferred detection method, nucleic acid amplicons are separated by size and/or charge, e.g., by electrophoresing the amplicons through an appropriate matrix (e.g., a polymer matrix such as polyacrylamide) and then detecting the nucleic acid with an appropriate detection reagent, such as a dye, a labeled probe, or the like. These methods are also commonly combined with blotting methods that transfer size/charge separated nucleic acids onto appropriate solid substrates (e.g., nylon) for Southern or northern blotting. Details regarding such methods are found in Sambrook and Ausubel.

Optionally, nucleic acid amplicons are removed from a microfluidic system and processed in accordance with standard methods such as those detailed in Sambrook and Ausubel. However, in an additional preferred embodiment, the microscale systems includes an integrated (or separate) electrophoretic detection channel, e.g., with a polymer matrix in the channel. The channel can be of microscale and can include integrated detectors. Microscale systems that include such integrated detection channels are commercially available, e.g., from Caliper Life Sciences, Inc. and Agilent Technologies. The various microfluidic references herein describe the integration of electrophoresis-based detection systems in some detail.

Solid Phase Detection—Array Based Detection

Amplicons made according to the present invention can be detected by any available method. As noted herein, this can include solution phase assays, including dye-incorporation, detection via FRET, molecular beacons, or TaqMan probes, detection of encoded products (discussed in more detail below), or the like. Such detection can also include any of a number of standard solid phase approaches, e.g., relying on hybridization of the nucleic acid amplicon to one or more probe, e.g., fixed on a solid phase. This can include standard Southern or northern blotting, southwestern or northwestern blotting (all described in detail in Sambrook and Ausubel) array-based hybridization, or the like. In one simple configuration, this can include extracting an amplicon from a product location (e.g., a well), using conventional or microfluidic fluid handling approaches (pipetting, robotic pipetting, flow through microfluidic systems, etc.) and loading it onto the appropriate solid phase (e.g., membranes, glass or plastic beads or slides) comprising one or more probe of interest.

Array based hybridization is particularly suitable for a number of applications, as it can be used for quantitatively measuring expression of many amplicons simultaneously. As noted throughout, one application of the continuous flow bioreactor of the invention is for expression profiling of nucleic acids of interest.

A number of array systems have been described and can be used in accordance with the present invention. One general example of laboratory tools utlizes arrays of biopolymers, such as arrays of nucleic acids or proteins. For example, companies such as Affymetrix (e.g., VLSIPS® arrays; Santa Clara, Calif.), Hyseq (Mountain View, Calif.), Research Genetics (e.g., the GeneFilters® microarrays; Huntsville Ala.), Axon Instruments (GenePix®; Foster City, Calif.), Operon (e.g., OpArrays®, Alameda, Calif.) and others provide many technologies for making physical arrays of nucleic acids and other molecules. For example, arrays have been used for Disease Management issues, Expression Analysis, GeneChip Probe Array Technologies, Genotyping and Polymorphism analysis, Spotted Array Technologies and the like. Reviews of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11 :A879; Fodor (1997) “Massively Parallel Genomics.” Science 277: 393-395; and Chee et al. (1996) “Accessing Genetic Information with High-Density DNA Arrays.” Science 274:610-614.

Non-Sequence Specific Detection of Product Nucleic Acids

In addition to the various dye-based approaches noted above, nucleic acids can be directly detected, e.g., using intrinsic fluorescence or absorbance (e.g., UV absorbance). These methods are generally well-taught with respect to standard cuvette/spectrophotometer based approaches in Sambrook and Ausubel. These cuvette based approaches can be used in the context of the present invention, e.g., by detecting RNA or DNA products via standard approaches using intrinsic fluorescence or absorbance. In addition, microfluidic approaches can also be used, in that fluorescence (e.g., total product fluorescence) or absorbance can be determined in microchannels or chambers via operably coupled detectors. These detectors, like the others noted herein, can be “on chip” or can be separate system components.

Detection of Amplified RNA by Detecting Encoded Products-Solution or Solid Phase Detection

In addition to approaches to detecting RNA or DNA directly, the invention also provides for detecting DNA or RNA by detecting encoded products (e.g., proteins encoded by a nucleic acid of interest). Once expressed, proteins or other polypeptide expression products can be purified and/or detected, either partially or substantially to homogeneity, according to standard procedures known to and used by those of skill in the art. These procedures can be adapted to microscale systems, but do not have to be, as one can alternatively collect proteins or RNA (or DNA) products and from output wells of a microscale system and analyze the products according to any available method. These purification methods include ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in completing configuration of mature proteins. High performance liquid chromatography (HPLC) can be employed in purification steps where high purity is desired. Once purified, partially or to homogeneity, as desired, the polypeptides may be used (e.g., as assay components, therapeutic reagents or as immunogens for antibody production).

Accordingly, proteins expressed according to the methods herein can be detected by standard solution phase assays, e.g., ELISA assays, or the like, or can be detected in a solid phase assay, e.g., via western blotting, or via array based detection. Protocols for western blotting and Elisa can be found in Ausubel, supra, as well as in Deutscher (1990), Sandana (1997); Bollag et al. (1996); Walker (1996); Harris and Angal (1990); Scopes (1993); Janson and Ryden (1998); and Walker (1998), all supra.

In one example, proteins are detected on arrays in a manner similar to nucleic acids, as noted above. Examples of protein-based arrays include immuno arrays are in Holt et al. (2000) “By-passing selection: direct screening for antibody-antigen interactions using protein arrays.” Nucleic Acids Research 28(15) E72-e72), superproteins arrays, yeast two and other “n” hybrid array systems (see, e.g. Uetz et al. (2000) “A comprehensive analysis of protein-protein interactions in Saccharomyces cerevisiae” Nature 403, 623-627, and Vidal and Legrain (1999) “Yeast forward and reverse ‘n’-hybrid systems.” Nucleic Acids Research 27(4) 919-929); the universal protein array or “UPA” system (Ge et al. (2000) “UPA, a universal protein array system for quantitative detection of protein-protein, protein-DNA, protein-RNA and protein-ligand interactions.” Nucleic Acids Research, 28(2): E3-e3) and the like. Commercial companies such as Ciphergen (Freemont, Calif.); www.ciphergen.com, Beckman Coulter Inc. (Brea, Calif. ); and others also provide commercial protein chip arrays.

Integrated Detectors

Amplification and detection are commonly integrated in a system comprising a microfluidic device in the present invention. Available microfluidic systems that include detection features for detecting nucleic acids include the LabChip® HTS system and the LabChip® 90 system from Caliper Life Sciences, Inc. (Mountain View, Calif.), as well as the Agilent 2100 bioanalyzer (Agilent, Palo Alto, Calif.). Additional details regarding systems that comprise detection (and separation/detection) capabilities are well described in the patent literature, e.g., the references already noted herein and in Parce et al. “High Throughput Screening Assay Systems in Microscale Fluidic Devices” WO 98/00231.

In general, the devices and systems herein optionally include signal detectors, e.g., which detect fluorescence, phosphorescence, radioactivity, pH, charge, absorbance, luminescence, temperature, magnetism or the like. Fluorescent detection is especially preferred and generally used for detection of amplified nucleic acids (however, upstream and/or downstream operations can be performed on amplicons, which can involve other detection methods).

The detector(s) optionally monitors one or a plurality of signals from an amplification reaction. For example, the detector can monitor optical signals which correspond to “real time” amplification assay results.

Example detectors include photo multiplier tubes, spectrophotometers, CCD arrays, scanning detectors, microscopes, galvo-scanns and/or the like. Amplicons or other components which emit a detectable signal can be flowed past the detector, or, alternatively, the detector can move relative to the site of the amplification reaction (or, the detector can simultaneously monitor a number of spatial positions corresponding to channel regions, or microtiter wells e.g., as in a CCD array).

The detector can include or be operably linked to a computer, e.g., which has software for converting detector signal information into assay result information (e.g., presence of a nucleic acid of interest), or the like.

Signals are optionally calibrated, e.g., by calibrating the microfluidic system by monitoring a signal from a known source.

A microfluidic system can also employ multiple different detection systems for monitoring a signal in the system. Detection systems of the present invention are used to detect and monitor the materials in a particular channel region (or other reaction detection region). Once detected, the flow rate and velocity of cells in the channels are also optionally measured and controlled as described above.

Examples of detection systems include optical sensors, temperature sensors, pressure sensors, pH sensors, conductivity sensors, and the like. Each of these types of sensors is readily incorporated into the microfluidic systems described herein. In these systems, such detectors are placed either within or adjacent to the microfluidic device or one or more channels, chambers or conduits of the device, such that the detector is within sensory communication with the device, channel, or chamber. The phrase “within sensory communication” of a particular region or element, as used herein, generally refers to the placement of the detector in a position such that the detector is capable of detecting the property of the microfluidic device, array or other relevant component a portion of the component, or the contents of a portion of the component, for which that detector was intended. For example, a pH sensor placed in sensory communication with a microscale channel is capable of determining the pH of a fluid disposed in that channel. Similarly, a temperature sensor placed in sensory communication with the body of a microfluidic device is capable of determining the temperature of the device itself. A fluoresecence detector is positioned to detect fluorescence from the relevant component (e.g., channel, chamber, array, etc.) of interest.

Particularly preferred detection systems include optical detection systems for detecting an optical property of a material within the channels and/or chambers of the microfluidic devices that are incorporated into the microfluidic systems described herein. Such optical detection systems are typically placed adjacent to a microscale channel of a microfluidic device, or an array or other relevant component, and are in sensory communication with the component via an optical detection window that is disposed across the component (e.g., channel or chamber of a microscale device, array, etc.). Optical detection systems include systems that are capable of measuring the light emitted from material within the component, the transmissivity or absorbance of the material, as well as the materials spectral characteristics. In preferred aspects, the detector measures an amount of light emitted from the material, such as a fluorescent or luminescent material. As such, the detection system will typically include collection optics for gathering a light based signal transmitted through the detection window, and transmitting that signal to an appropriate light detector. Microscope objectives of varying power, field diameter, and focal length are readily utilized as at least a portion of this optical train. The light detectors are optionally spectrophotometers, photodiodes, avalanche photodiodes, photomultiplier tubes, diode arrays, or in some cases, imaging systems, such as charged coupled devices (CCDs) and the like. The detection system is typically coupled to a computer, via an analog to digital or digital to analog converter, for transmitting detected light data to the computer for analysis, storage and data manipulation.

In the case of fluorescent materials such as labeled aRNA, the detector typically includes a light source that produces light at an appropriate wavelength for activating the fluorescent material, as well as optics for directing the light source through the detection window to the material contained in the channel or chamber. The light source can be any number of light sources that provides an appropriate wavelength, including lasers, laser diodes and LEDs. Other light sources are used in other detection systems. For example, broad band light sources are typically used in light scattering/transmissivity detection schemes, and the like. Typically, light selection parameters are well known to those of skill in the art.

The detector can exist as a separate unit, but can also be integrated with the system or microfluidic device, into a single instrument. Integration of these functions into a single unit facilitates connection of these instruments with the computer, by permitting the use of few or a single communication port(s) for transmitting information between the controller, the detector and the computer.

Accordingly, the systems of the invention can include microfluidic devices, detectors, sample storage elements (microtiter plates, dried arrays of components, etc.), flow controllers, amplification devices or other microfluidic modules, computers and/or the like. These systems can be used for aliquoting, amplifying and analyzing the nucleic acids of interest. The microfluidic devices, amplification components, detectors and storage elements of the systems have already been described in some detail above. The following discussion describes various appropriate controllers and computers, though many configurations are available and one of skill would be expected to be familiar in their use and would understand how they can be applied to the present invention.

Flow Controllers

A variety of controlling instrumentation is optionally utilized in conjunction with the microfluidic devices described herein, for controlling the transport and direction of fluids and/or materials within the devices of the present invention, e.g., by pressure-based or electrokinetic control. For example, reagents for RNA amplification, or RNA amplicons can be processed according to any method herein, e.g., by flowing the reagents or amplicons into contact with one or more additional component of any relevant method.

For example, in many cases, fluid transport and direction are controlled in whole or in part, using pressure based flow systems that incorporate external or internal pressure sources to drive fluid flow. Internal sources include microfabricated pumps, e.g., diaphragm pumps, thermal pumps, Lamb wave pumps and the like that have been described in the art. See, e.g., U.S. Pat. Nos. 5,271,724, 5,277,556, and 5,375,979 and Published PCT Application Nos. WO 94/05414 and WO 97/02357.

The systems described herein can also utilize electrokinetic material direction and transport systems. Details regarding electrokinetic flow is found throughout the relevant technical and patent literature. A variety of electrokinetic controllers are described, e.g., in Ramsey WO 96/04547, Parce et al. WO 98/46438 and Dubrow et al., WO 98/49548, as well as a variety of other references noted herein. See also, Ramsey et al. (1995), Nature Med. 1(10):1093-1096; Kopf-Sill et al. (1997) “Complexity and performance of on-chip biochemical assays,” SPIE 2978:172-179 February 10-11; Bousse et al. (1998) “Parallelism in integrated fluidic circuits,” SPIE 3259:179-186; Chow et al. U.S. Pat. No. 5,800,690; Kopf-Sill et al. U.S. Pat. No. 5,842,787; Parce et al., U.S. Pat. No. 5,779,868; Parce, U.S. Pat. No. 5,699,157; U.S. Pat. No. 5,852,495 (J. Wallace Parce) issued Dec. 22, 1998; U.S. Pat. No. 5,869,004 (J. Wallace Parce et al.) issued Feb. 09, 1999, U.S. Pat. No. 5,876,675 (Colin B. Kennedy) issued Mar. 02, 1999; U.S. Pat. No. 5,880,071 (J. Wallace Parce et al.) issued Mar. 09, 1999; U.S. Pat. No. 5,882,465 (Richard J. McReynolds) issued Mar. 16, 1999; U.S. Pat. No. 5,885,470 ( J. Wallace Parce et al.) issued Mar. 23, 1999; U. S. Pat. No. 5,942,443 (J. Wallace Parce et al.) issued Aug. 24, 1999; U.S. Pat. No. 5,948,227 (Robert S. Dubrow) issued Sep. 07, 1999; U.S. Pat. No. 5,955,028 (Calvin Y. H. Chow) issued Sep. 21, 1999; U.S. Pat. No. 5,957,579 (Anne R. Kopf-Sill et al.) issued Sep. 28, 1999; U.S. Pat. No. 5,958,203 (J. Wallace Parce et al.) issued Sep. 28, 1999; U.S. Pat. No. 5,958,694 (Theo T. Nikiforov) issued Sep. 28, 1999; and U.S. Pat. NO. 5,959,291 ( Morten J. Jensen) issued Sep. 28, 199; Parce et al. WO 98/00231; Parce et al. WO 98/00705; Chow et al. WO 98/00707; Parce et al. WO 98/02728; Chow WO 98/05424; Parce WO 98/22811; Knapp et al., WO 98/45481; Nikiforov et al. WO 98/45929; Parce et al. WO 98/46438; Dubrow et al., WO 98/49548; Manz, WO 98/55852; WO 98/56505; WO 98/56956; WO 99/00649; WO 99/10735; WO 99/12016; WO 99/16162; WO 99/19056; WO 99/19516; WO 99/29497; WO 99/31495; WO 99/34205; WO 99/43432; and WO 99/44217; U.S. Pat. No. 5,296,114; and e.g., EP 0 620 432 A1; Seiler et al. (1994) Mitt Gebiete Lebensm. Hyg. 85:59-68; Seiler et al. (1994) Anal. Chem. 66:3485-3491; Jacobson et al. (1994) “Effects of Injection Schemes and Column Geometry on the Performance of Microchip Electrophoresis Devices” Anal. Chem. 66: 66. 1107-1113; Jacobsen et al. (1994) “Open Channel Electrochromatograpy on a Microchip” Anal. Chem. 66:2369-2373; Jacobsen et al. (1994) “Precolumn Reactions with Electrophoretic Analysis Integrated on Microchip” Anal. Chem. 66:4127-4132; Jacobsen et al. (1994) “Effects of Injection Schemes and Column Geometry on the Performance of Microchip Electrophoresis Devices.” Anal. Chem. 66:1107-1113; Jacobsen et al. (1994) “High Speed Separations on a Microchip.” Anal. Chem. 66:1114-1118; Jacobsen and Ramsey (1995) “Microchip electrophoresis with sample stacking” Electrophoresis 16:481-486; Jacobsen et al. (1995) “Fused Quartz Substrates for Microchip Electrophoresis” Anal. Chem. 67: 2059-2063; Harrison et al. (1992) “Capillary Electrophoresis and Sample Injection Systems Integrated on a Planar Glass Chip.” Anal. Chem. 64:1926-1932; Harrison et al. (1993) “Micromachining a Miniaturized Capillary Electrophoresis-Based Chemical Analysis System on a Chip.” Science 261: 895-897; Harrison and Glavania (1993) “Towards Miniaturized Electrophoresis and Chemical System Analysis Systems on Silicon: An Alternative to Chemical Sensors.” Sensors and Actuators 10:107-116; Fan and Harrison (1994) “Micromachining of Capillary Electrophoresis Injectors and Separators on Glass Chips and Evaluation of Flow at Capillary Intersections. Anal. Chem. 66: 177-184; Effenhauser et al. (1993) “Glass Chips for High-Speed Capillary Electrophoresis Separations with Submicrometer Plate Heights” Anal. Chem. 65:2637-2642; Effenhauser et al. (1994) “High-Speed Separation of Antisense Oligonucleotides on a Micromachined Capillary Electrophoresis Device.” Anal. Chem. 66:2949-2953; and Kovacs EP 0376611 A2. Kopp et al. (1998) “Chemical Amplification: Continuous Flow PCR on a Chip” Science, 280 (5366):1046; U.S. Pat. No. 6,444,461 to Knapp, et al. (Sep. 3, 2002) MICROFLUIDIC DEVICES AND METHODS FOR SEPARATION; U.S. Pat. No. 6,406,893 to Knapp, et al. (Jun. 18, 2002) MICROFLUIDIC METHODS FOR NON-THERMAL NUCLEIC ACID MANIPULATIONS; U.S. Pat. No. 6,391,622 to Knapp, et al. (May 21, 2002) CLOSED-LOOP BIOCHEMICAL ANALYZERS; U.S. Pat. No. 6,303,343 to Kopf-Sill (Oct. 16, 2001) INEFFICIENT FAST PCR; U.S. Pat. No. 6,171,850 to Nagle, et al. (Jan. 9,2001) INTEGRATED DEVICES AND SYSTEMS FOR PERFORMING TEMPERATURE CONTROLLED REACTIONS AND ANALYSES; U.S. Pat. No. 5,939,291 to Loewy, et al. (Aug. 17, 1999) MICROFLUIDIC METHOD FOR NUCLEIC ACID AMPLIFICATION; U.S. Pat. No. 5,955,029 to Wilding, et al. (Sep. 21, 1999) MESOSCALE POLYNUCLEOTIDE AMPLIFICATION DEVICE AND METHOD; U.S. Pat. No. 5,965,410 to Chow, et al. (Oct. 12, 1999) ELECTRICAL CURRENT FOR CONTROLLING FLUID PARAMETERS IN MICROCHANNELS; and the references cited therein.

In some embodiments, external pressure sources are used, and applied to ports at channel termini. These applied pressures (which can be positive or negative (vacuum) pressure), generate pressure differentials across the lengths of channels to drive fluid flow through them. In the interconnected channel networks described herein, differential flow rates on volumes are optionally accomplished by applying different pressures or vacuums at multiple ports, or preferably, by applying a single vacuum at a common waste port and configuring the various channels with appropriate resistance to yield desired flow rates. Example systems are described in U.S. Ser. No. 09/238,467 filed Jan. 28, 1999.

Typically, the controller systems are appropriately configured to receive or interface with a microfluidic device or system element as described herein. For example, the controller and/or detector, optionally includes a stage upon which a microfluidic device is mounted to facilitate appropriate interfacing between the controller and/or detector and the device. Typically, the stage includes an appropriate mounting/alignment structural element, such as a nesting well, alignment pins and/or holes, asymmetric edge structures (to facilitate proper device alignment), and the like. Many such configurations are described in the references cited herein.

The controlling instrumentation discussed above is also optionally used to provide for electrokinetic injection or withdrawal of material downstream of the region of interest to control an upstream flow rate. The same instrumentation and techniques described above are also utilized to inject a fluid into a downstream port to function as a flow control element.

In several embodiments herein, reagents are flowed across bead beds. Details regarding devices that incorporate such features are found, e.g., in Burd Mehta et al. (2000), MANIPULATION OF MICROPARTICLES IN MICROFLUIDIC SYSTEMS, WO 00/50172. Methods of making bead beds within microscale devices are provided in WO 00/50172.

Combinations of Electrokinetic and Pressure Based flow Control—Example Embodiment.

As noted above, pressure-based or electrokinetic based flow control (or both) can be used in the present invention. In one embodiment, both are used, serially or simultaneously, to produce desireable results. In this embodiment, pressure based flow control provides a relatively simple and effective method of controlling flow for delivery of assay components (e.g., beads, amplification reagents or the like) to a reaction chamber. Flow out of the chamber advantageously uses electrokinetic flow in certain applications, taking advantage of the highly charged nature of nucleic acid amplicons. That is, the highly charged nature of nucleic acid amplicons results in fast flow of the amplicons when an electric field is applied, due to electrophoretic (and, to some extent, electroosmotic) forces. Pressure based flow can also be used simultaneously, or in series with, such electrokinetic forces, to further drive product or reagent movement into, through, or out of a reaction chamber, e.g., a chamber comprising a reaction bead bed.

Non-Continuous Flow Based Applications

In one aspect, a non-continuous flow based approach is used. In this approach, reactants are flowed into a reaction chamber (which can be microscale or non-microscale), e.g., though channels (which can be microscale or non-microscale). These reactants are reacted in the reaction chamber, partly or fully to completion. Products are then flowed out the chamber through the same or different channels (which can be microscale or non-microscale). As discussed above, continuous or semi-continuous flow applications have several advantages with respect to system optimization. However, non-continuous flow applications can also be used and may have certain advantages, e.g., eliminating the need for constant flow optimization. In these embodiments, the flow controller is configured for stopped flow during some or all of the reaction.

Computer

As noted above, either or both of the controller system and/or the detection system are coupled to an appropriately programmed processor or computer which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation. The computer then receives the data from the one or more sensors/detectors included within the system, and interprets the data, either provides it in a user understood format, or uses that data to initiate further controller instructions, in accordance with the programming, e.g., such as in monitoring and control of flow rates (including for continuous flow), temperatures, applied voltages, and the like. In accordance with the present invention, these parameters are optimized for production of nucleic acid amplicons such as RNAs.

The systems and/or kits can include system instructions (e.g., embodied in a computer or in a computer readable medium, e.g., as system software) for practicing any of the method steps herein. For example, the system optionally includes system software that directs the system to perform any of the method steps noted above, e.g., in one embodiment, the system includes instructions for performing a Van Gelder-Eberwine series of reactions that converts one or more starting RNA into DNA by reverse transcription, performs a second strand synthesis to produce double stranded DNA and transcribes the double stranded DNA to produce the RNA amplicons. For example, the instructions can direct the various temperature and flow controllers to flow appropriate materials from sources to reaction sites and/or to control reaction conditions (e.g., temperature) at the sites.

In the present invention, the computer typically includes software for the monitoring of materials in the channels. Additionally, the software is optionally used to control electrokinetic or pressure modulated injection or withdrawal of material. The injection or withdrawal is used to modulate the flow rate as described above, to mix components, and the like.

Example System

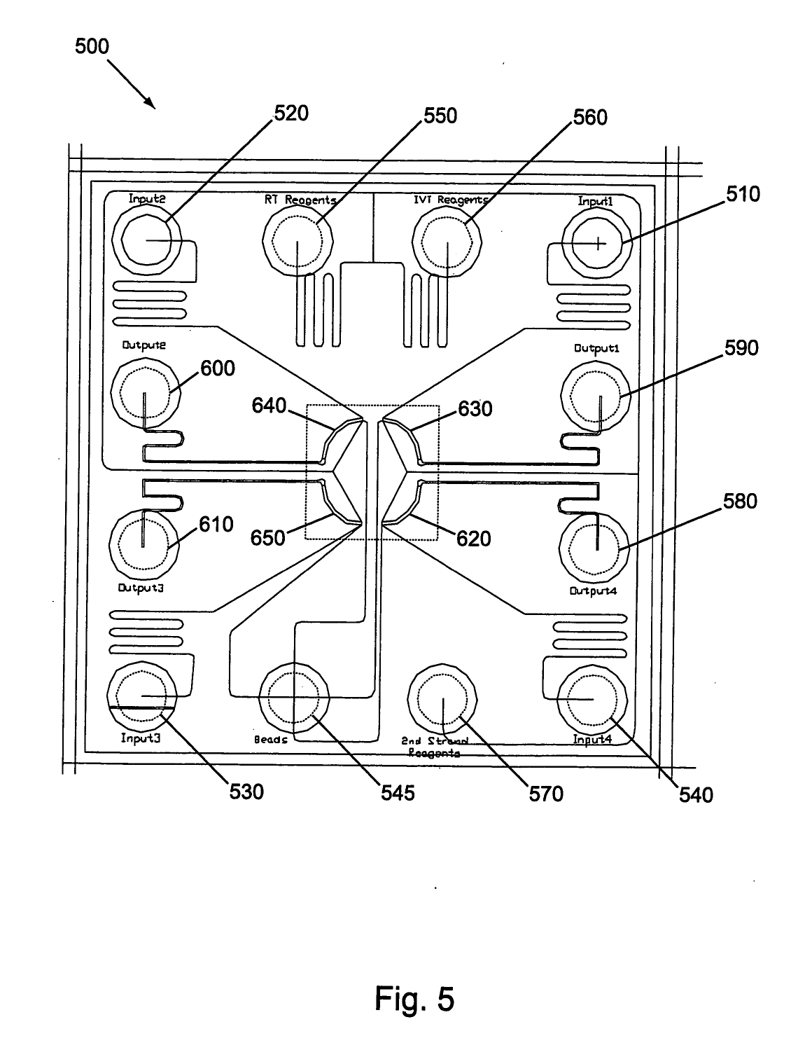

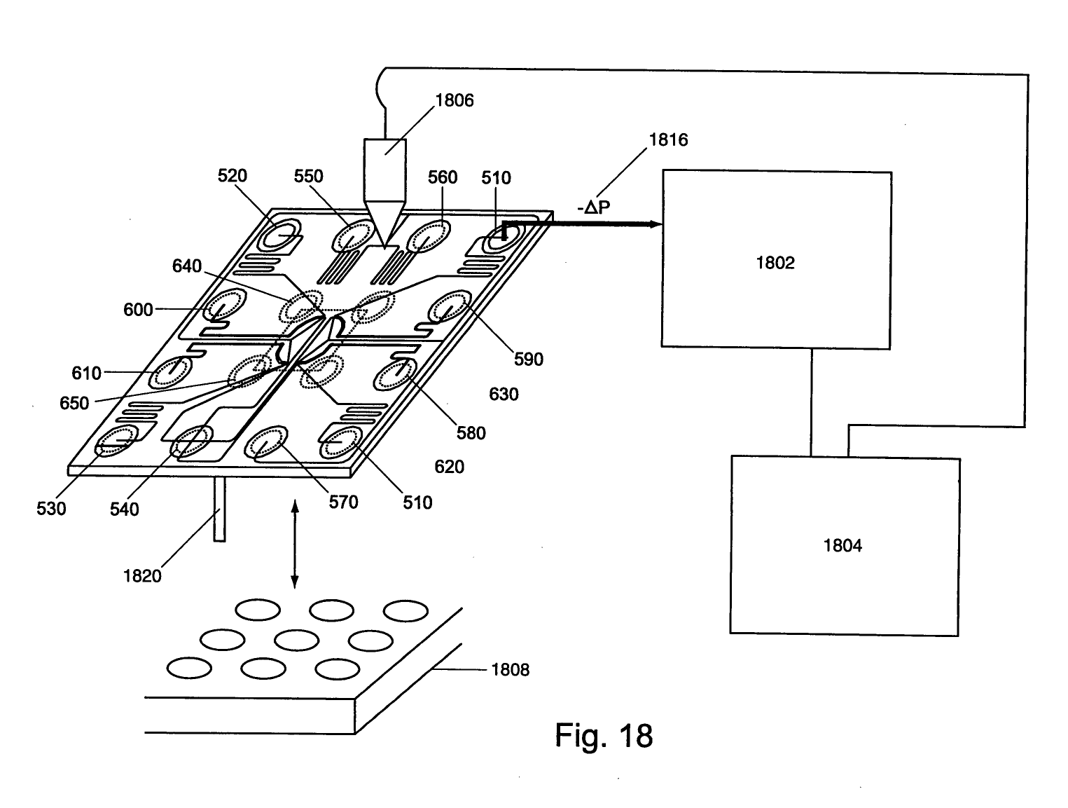

FIG. 18 provides a schematic illustration of a model system of the invention. As shown, device 500 (in this example, a device similar to that of FIG. 5, except for the inclusion of an optional pipettor element, is used for clarity of illustration in the system, but it will be appreciated that any device as described herein can be alternatively be used in a system of the invention) As illustrated, device 500 includes input wells 510-540, as well as bead source well 540 and the reagents needed for an amplification reaction such as the Van Gelder Eberwine reaction in reverse transcriptase reagent source well 550, in vitro transcription reagent well 560 and second strand reagent well 570. Output wells are illustrated in wells 580-610. Reaction chambers 620-650 are also illustrated. This device design permits 4 reactions to be performed simultaneously. The arrangement of elements on the device is optimized to reduce the size of the device and to reduce the number of pumps that drive material transport on the chip, thereby reducing instrumentation costs. The chip features 4 reaction chambers (620-650), four input source material wells (510-540) four output material container wells (580-610), three reagent source wells (550-570) and one bead source well (545). Device 500 couples to pressure source 1816 at the various wells. The pressure source can conveniently run multiple wells, e.g., 1 source can couple to input wells 510-540, while another source couples to output wells 580-610. Typically, the reagent and bead wells will be coupled to separate pressure sources. One of skill will understand that the number of pressure sources (e.g., pumps) can be increased or decreased by using more or fewer sources coupled to more or fewer wells through appropriate couplings.

In operation, amplification components are flowed by applying a vacuum at vacuum source 1816 (and/or at any of the reservoirs or wells noted herein) through reaction chambers 620-650. Amplification components can also be flowed from wells 510-540 and into chambers 620-650. Materials can also be flowed into these wells, e.g., when they are used as waste wells, or when they are coupled to a vacuum source. Flow from wells can be performed by modulating fluid pressure, or by electrokinetic approaches, or both. Instead of the arrangement of channels depicted in FIGS. 5, 6 and 18, an arrangement such as the device of FIGS. 15-17, or any other herein can be substituted. In addition, a variety of other appropriate microfluidic configurations are set forth in the references noted herein and can be adapted to the present invention by configuring the devices as noted herein.

Materials relevant to performing the amplification reactions can be flowed from the enumerated wells, or can be flowed from a source external to device 500. As depicted, the integrated system can include pipettor channel 1820, e.g., protruding from device 500, for accessing an outside source of reagents. For example, as further depicted optional pipettor channel 1820 can access microwell plate 1808 which includes samples or sample aliquots, or locus specific reagents, or other reagents useful in the practice of the invention in the wells of the plate. Aliquots or reagents relevant to amplification can be flowed into any of the channels of device 500 through optional pipettor channel 1820. Detector 1806 is optionally in sensory communication with one or more channels of the device (or integral or separate detection components, such as one or more nucleic acid detection arrays), detecting signals resulting, e.g., from the interaction of a label with an amplicon as described herein. Detector 1806 is optionally operably linked to Computer 1804, which digitizes, stores and manipulates signal information detected by detector 1806.

Voltage/pressure controller 1802 controls voltage, pressure, or both, e.g., at the wells of the system, or at vacuum couplings fluidly coupled to the channels of device 500. Optionally, as depicted, computer 1804 controls voltage/pressure controller 1802. In one set of embodiments, computer 1804 uses signal information to select further reaction parameters. For example, upon detecting amplification of a nucleic acid of interest, the computer optionally directs withdrawal of additional aliquots from one or more well (of device 500 or plate 1808, or both) for analysis, e.g., through pipettor channel 1802, e.g., to deliver different concentrations of the aliquot to the amplification reaction. If statistical or quantitative information is desired, computer 1804 directs controller 1802 to perform appropriate fluid manipulations to generate data for the statistical or quantitative analysis. Computer 1804 is optionally coupled to or comprises a user viewable display, permitting control of the computer by the user and providing a readout for the user to view results detected by the system.

Additional Kits Details

The present invention also provides kits for carrying out the methods described herein. In particular, these kits typically include system components described herein, as well as additional components to facilitate the performance of the methods by an investigator.

The kit also typically includes a receptacle in which the system component is packaged. The elements of the kits of the present invention are typically packaged together in a single package or set of related packages. The package optionally includes reagents used in the assays herein, e.g., buffers, amplification reagents, standard reagents, and the like, as well as written instructions for carrying out the assay in accordance with the methods described herein. In the case of prepackaged reagents, the kits optionally include pre-measured or pre-dosed reagents that are ready to incorporate into the methods without measurement, e.g., pre-measured fluid aliquots, or pre-weighed or pre-measured solid reagents that may be easily reconstituted by the end-user of the kit.

Generally, the microfluidic devices described herein are optionally packaged to include reagents for performing the device's preferred function. For example, the kits can include any of microfluidic devices described along with assay components, reagents, sample materials, control materials, or the like. Such kits also typically include appropriate instructions for using the devices and reagents, and in cases where reagents are not predisposed in the devices themselves, with appropriate instructions for introducing the reagents into the channels and/or chambers of the device. In this latter case, these kits optionally include special ancillary devices for introducing materials into the microfluidic systems, e.g., appropriately configured syringes/pumps, or the like (in one preferred embodiment, the device itself comprises a pipettor element, such as an electropipettor for introducing material into channels and chambers within the device). In the former case, such kits typically include a microfluidic device with necessary reagents predisposed in the channels/chambers of the device. Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (i.e., enzymatic inhibitors, microcides/bacteriostats, anticoagulants), the physical stabilization of the material, e.g., through immobilization on a solid support, entrapment in a matrix (i.e., a gel), lyophilization, or the like.

### EXAMPLES

The following examples are intended to be illustrative, but not limiting. One of skill will immediately recognize a variety of non-critical parameters that can be altered.

Continuous Flow Bioreactors

Continuous flow bioreactors of this example are designed to operate in a continuous flow, temperature controlled mode. The bioreactors of this example are microscale devices in which reagents are delivered in a continuous flow format for on-device, temperature controlled enzymatic reactions. The reactions are conducted in microscale chambers (in this case channels) of the microscale devices. This is in contrast to prior art RNA amplification reactions, which do not, ordinarily, utilize continuous reagent replacement to keep a reaction going indefinitely. In addition, the reactor of this example overcomes products inhibition effects (e.g., in the case of RNA amplification, sense suppression effects occur as product is produced). This inhibition is overcome with the reactors of this example because products are continuously flowed out of the reaction chamber, preventing buildup of products. Product detection can be performed in the device, by labeling some or all of the product RNA e.g., with RNA specific fluorescent dyes, and detected. This can be performed in real time. Alternately, the RNA can simply be collected, e.g., from a product well into which it is flowed, and processed according to standard methods (e.g., array hybridization or northern analysis).

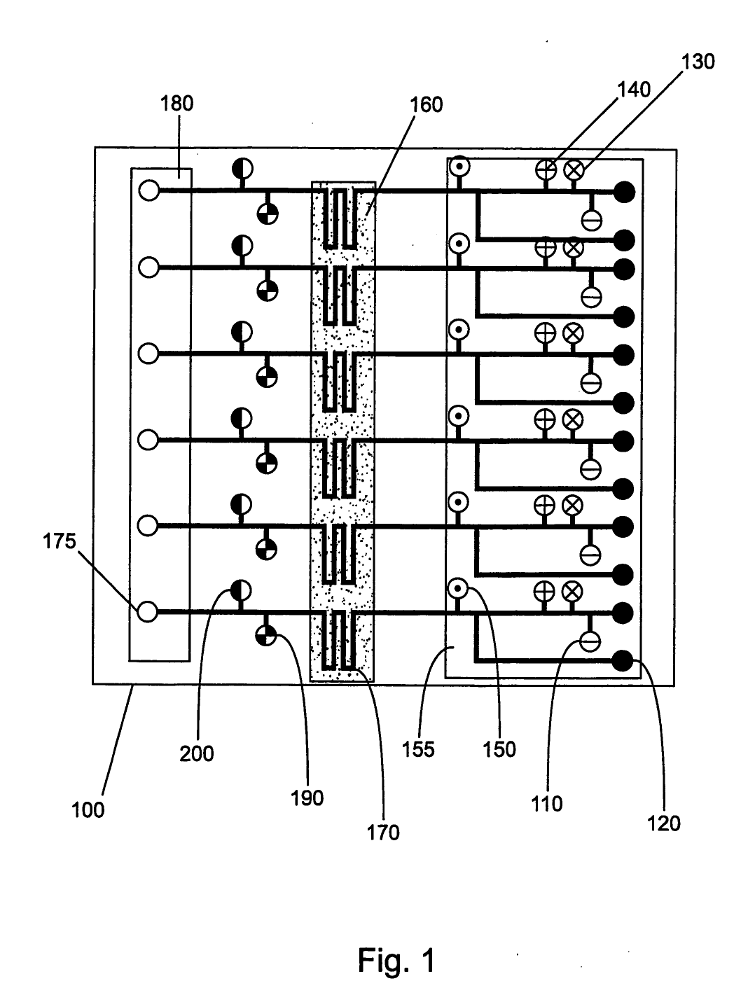

FIG. 1 schematically illustrates an example microscale device adapted to use in the present invention. As shown, device 100 comprises RNA synthesis reagent wells 110-150 in cooled zone 155. Reagents are flowed in a continuous fashion into heated zone 160 comprising reaction chamber 170. As depicted, the reaction chamber is simply a channel region in the appropriate temperature zone, but it can take other forms, such as widened or narrowed channel regions, or the like. Reaction products are optionally stored in and/or collected from product wells 175 in cooled zone 180. The device optionally comprises separate waste wells 190-200.

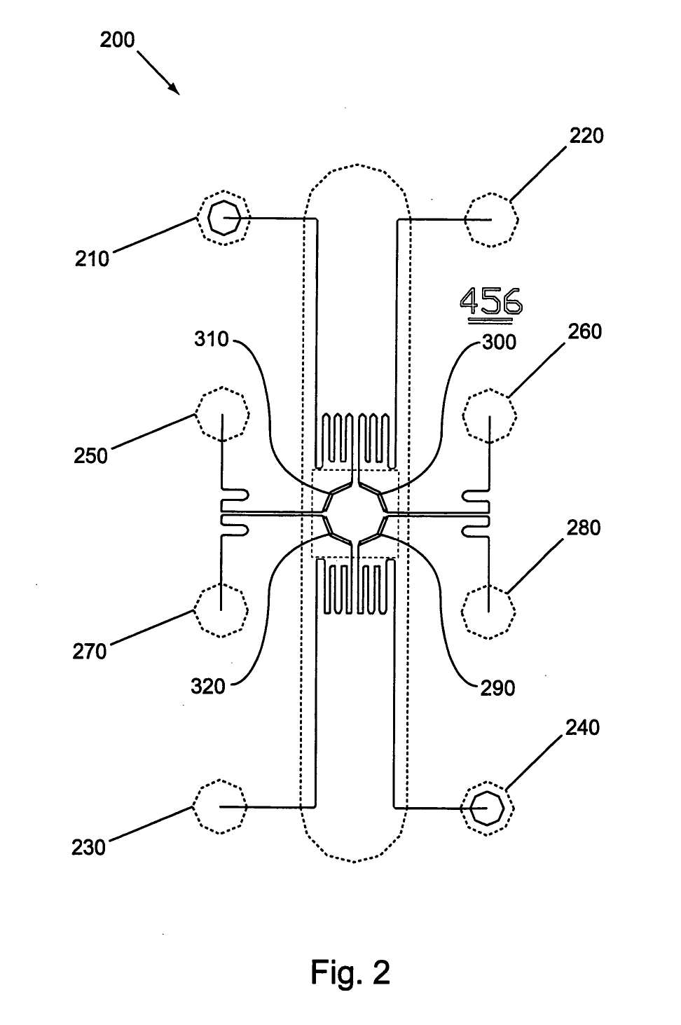

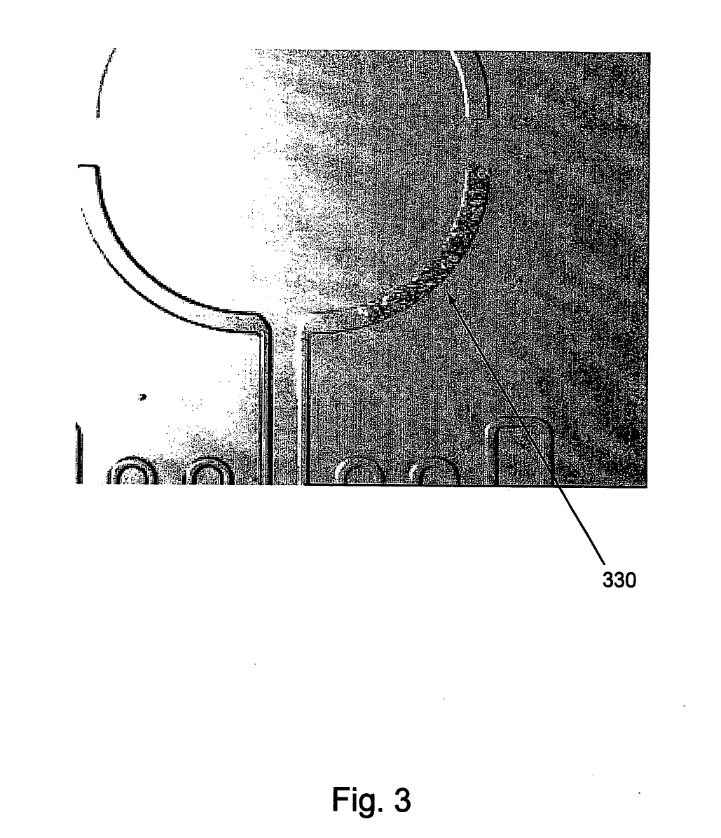

FIG. 2 schematically illustrates a second example device adapted to use in the present invention. Device 200 comprises input wells 210-240 and output wells 250-280. The device also includes reaction chambers 290-320. Beads comprising reaction components can be laid down in bead beds 330 illustrated in a photomicrograph of a device constructed according to the device of FIG. 2 (see, FIG. 3).

Table 1 illustrates various conditions used in the device of FIGS. 2-3:

TABLE 1

| | | | | Superficial |
|---|---|---|---|---|
| | Flow Rate | Flow Rate | Volume over | Velocity |
| ΔP (psi) | (nL/s) | (μL/hour) | 16 hours (μL) | (mm/s) |
| 1 | 18.22 | 1.09 | 17.49 | 0.15 |
| 2 | 36.43 | 2.19 | 34.98 | 0.30 |
| 3 | 54.65 | 3.28 | 52.47 | 0.45 |
| 4 | 72.87 | 4.37 | 69.95 | 0.60 |
| 5 | 91.09 | 5.47 | 87.44 | 0.75 |
| 6 | 109.30 | 6.56 | 104.93 | 0.90 |
| 10 | 182.17 | 10.93 | 174.89 | 1.51 |

Conditions:
Viscosity = 1 cP
Bead Diameter = 10 μm
Porosity = 0.6→ Packing Density = 40%
Miscellaneous specifications: well volume = 25 μL;
Reaction Chamber volume = 7944690.05 μm³
10 μm bead volume = 523.6 μm³
number of beads (with packing density of 40%) = 6069.

In an experimental use of the device of FIGS. 2-3, 10 μm beads were loaded into two reaction chambers. IVT buffer was allowed to flow through reaction chambers 290-320, where two of the chambers included bead bed 330 and two did not (i.e., accounting for all four circuits illustrated in FIG. 2). After 30 minutes, the volume of fluid in the input and output wells was measured to estimate the flow rate through reaction chambers 290-320. The results are illustrated in

TABLE 2

| Circuit | Volume Collected (μL) | Bead Bed? |
|---|---|---|
| 1 | 5 | Yes |
| 2 | 4.75 | Yes |
| 3 | 6 | No |
| 4 | 6.1 | No |

These results show that the hydrodynamic resistance of the bead bed for each circuit is much less than the resistance of the output feed channel: R_{bead bed}=5.44⁹ (g/cm⁴s), vs. R_{output feed}=2.16¹¹ (g/cm⁴s).

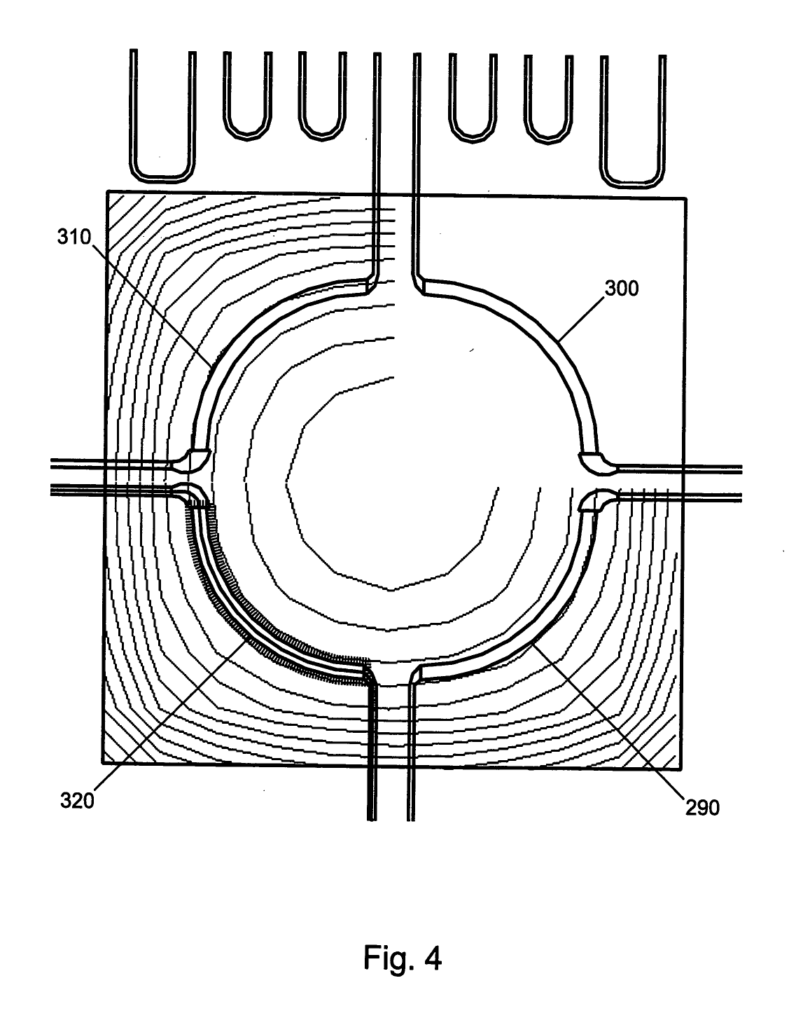

A computational model of the isothermal profile of the reaction chamber was performed, as illustrated in FIG. 4. As illustrated, FIG. 4 shows a computer model of heat transfer and the resulting isothermal counters as shown for the device of FIGS. 2-3. As illustrated in FIG. 4, reaction chambers 290-330 is curved to follow the predicted isotherms.

A calculation to determine how long the fluid in the chamber takes to reach the same temperature as the reaction chamber, upon entering the reaction chamber was performed. This was determined by determining how much warming distance the fluid needs to be pre-warmed upon entering the chamber (to prevent temperature effects in the chambers). For water at 300K:ρ:=1000 Kg(m³)⁻¹; Cp:=4190 J(Kg*K)⁻¹; K:=0.6 W(m*K)⁻¹. The distance from the top to the center of the channel (x) is calculated as X:=15(10⁻⁶ m); τ=(ρCp/K)x². Therefore, 4τ=6.285(10⁻³)s. This means that it will take about 6.3 ms for the temperature at the center of the channel to reach 99% of the temperature along the top edge of the channel.

A calculation of showing that the fluid flow will not appreciably cool the reaction chamber was also performed. For the calculation, depth of the example chamber depth (d) was 30(10⁻⁶)m, width (w) was 100(10⁻⁶)m, length (l) was 1.8 (10⁻³)m. Tf:=350 K; Ti:=300 K. Q:=Cp(dwl)ρ(Tf−Ti)=1.131 (10⁻³)J. If a flow velocity of 1 mm/s is assumed, then the power carried away by the fluid is: Q(1/s)=1.131(10⁻³)W.

FIG. 5 schematically illustrates an additional example microscale device according to the present invention. As illustrated, device 500 includes input wells 510-540, as well as bead source well 540 and the reagents needed for the Van Gelder Eberwine reaction in reverse transcriptase reagent source well 550, in vitro transcription reagent well 560 and second strand reagent well 570. Output wells are illustrated in wells 580-610. Reaction chambers 620-650 are also illustrated. This device design permits 4 Van Gelder Eberwine reactions to be performed simultaneously. The arrangement of elements on the device is optimized to reduce the size of the device and to reduce the number of pumps that drive material transport on the chip, thereby reducing instrumentation costs. The chip features 4 reaction chambers (620-650), four input source material wells (510-540) four output material container wells (580-610), hree reagent source wells (550-570) and one bead source well (545). Device 500 couples to pressure sources at the various wells. The pressure sources can conveniently run multiple wells, e.g., 1 source can couple to input wells 510-540, while another source couples to output wells 580-610. Typically, the reagent and bead wells will be coupled to separate pressure sources. One of skill will understand that the number of pressure sources (e.g., pumps) can be increased or decreased by using more or fewer sources coupled to more or fewer wells through appropriate couplings.

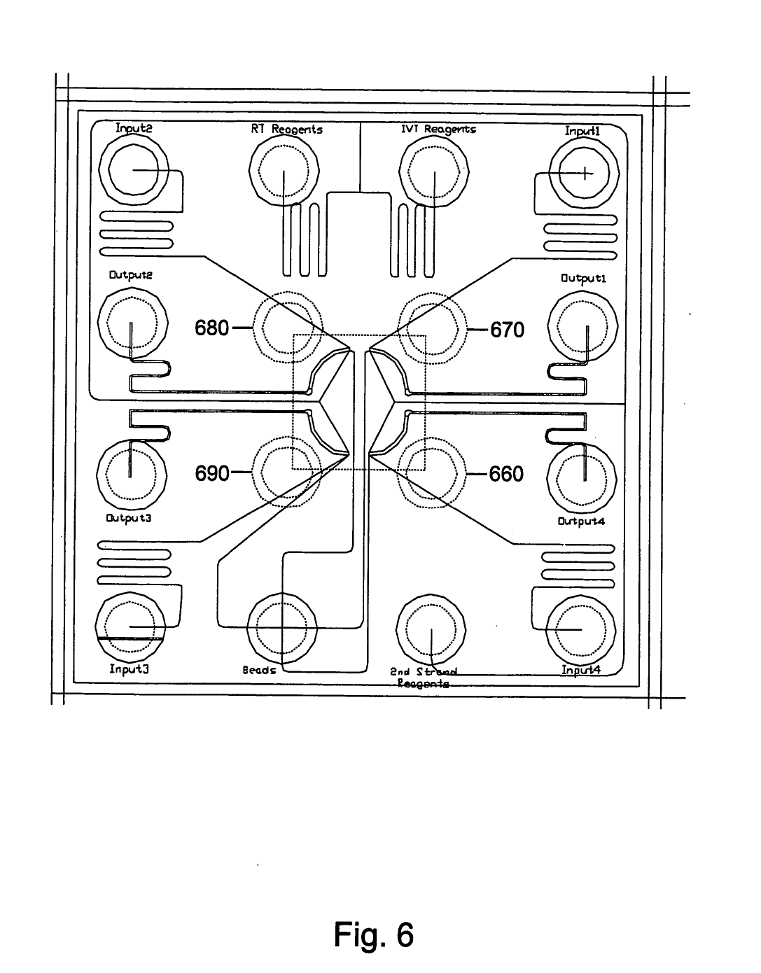

FIG. 6 illustrates a variation of the device of FIG. 5 that comprises 4 additional wells 660-690. These additional wells can be used to run two additional reactions, or can be used for an on-device RNA purification module.

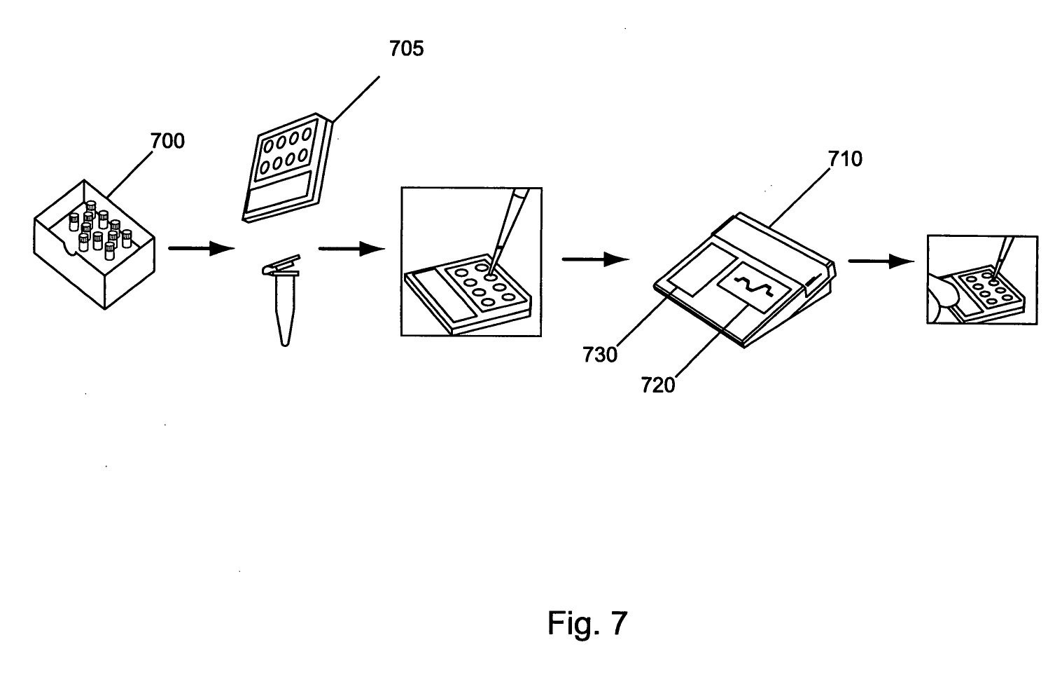

FIG. 7 illustrates an example system of the invention that can use, e.g., the devices of FIGS. 1-6. As illustrated, kit 700 comprises some or all of the reagents, buffers and enzymes used in a microscale device of the invention, e.g., as illustrated above. The kit optionally also includes the microscale device itself (schematically illustrated as device 705). As illustrated, the components are in appropriate containers, e.g., with appropriate packaging materials. The relevant RNA sample is prepared, as is the chip, for use on the device. The RNA sample is loaded along with the kit reagents onto the microscale device. The microscale device is loaded into instrument 710 that has the appropriate pressure and/or electrokinetic couplings to move fluids on the device and to apply heating or cooling to the microscale device. As illustrated, instrument 710 can include touch pad 720 to start the machine or to program in user-desired operations. Lid 730 covers device 705 during operation of the instrument. Amplified RNA is removed from appropriate wells of device 705, ready for purification, concentration, Q/C analysis, microarray use or any other purpose desired. This system replaces manually-intensive methods with an automated integrated system that provides highly reproducible amplification from sample to sample, and experiment to experiment, as well as providing standardized processes and a dramatic reduction in the time needed to perform the steps of the reactions involved (e.g., to about 8 hours, from 3 days, with most of the 8 hours being unattended operation).

The devices or systems can also include RNA purification modules, RNA extraction modules (e.g., to extract RNA direct from tissues of interest), cDNA labeling, in vitro transcription, in vitro translation, RT-PCR, RNA fragmentation and/or integrated Q/C components.

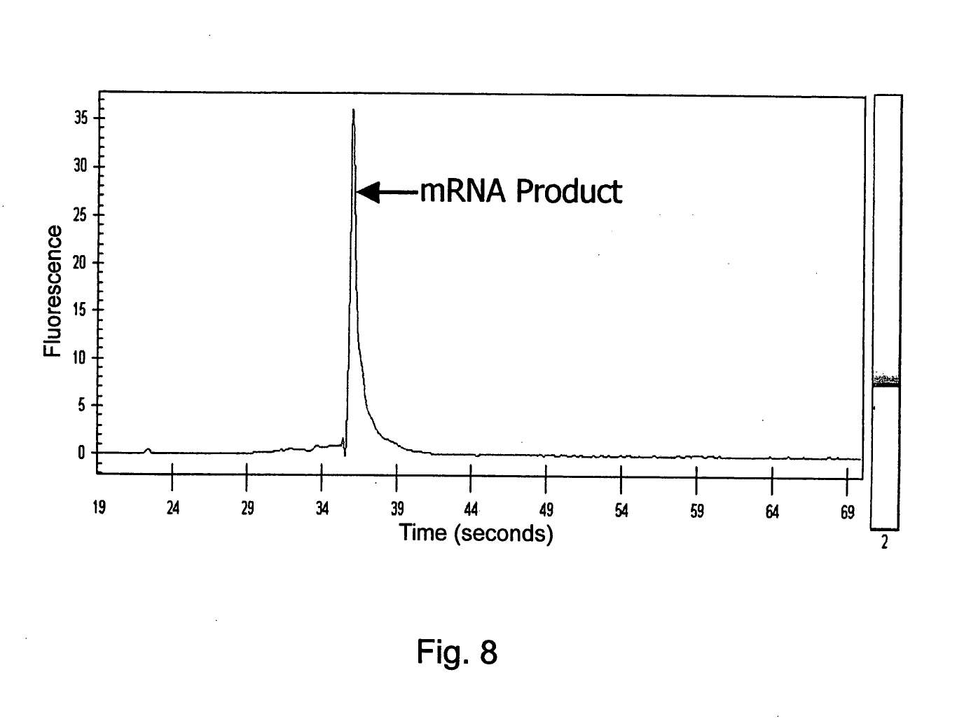

The data displayed in FIG. 8 shows the results from a continuous flow IVT reaction performed inside a NS456, under the following conditions. First, double stranded DNA template was prepared by synthesizing biotinilated-T7 promoter onto XEF transcript (about 1000 bp long). The dsDNA templates were bound to streptavadin coated, 10 um diameter, polystyrene microspheres (Bangs Labs), via a biotin-streptavadin linkage. Approximately 4000 of the template covered beads were loaded into the reaction chamber of microfluidic reactor of FIG. 5. 10 uL of In Vitro Transcription reagents (Ambion, Inc MegaScript kit) was loaded into an input well of the chip, and flowed through the reaction chamber at a flow rate of about 4 ul/hour (pressure gradient between input and output wells=4 psi). The reaction progressed at room temperature for 1 hour, and the reaction product was collected. The reaction was then allowed to continue for another hour with the reaction chamber heated to 37C. The reaction product was analyzed using a Bioanalyzer-2100 (Agilent Technologies) to assay the quality and yield quantity, and to compare the yield with heating vs. room temperature. FIG. 8 illustrates results from a successful amplification of mRNA on a bead-based microscale device as above. As illustrated, an analysis demonstrated that mRNA product was produced. The expected size of the RNA was 1000 bp, which was the same as the RNA obtained, with no evidence of RNA degradation.

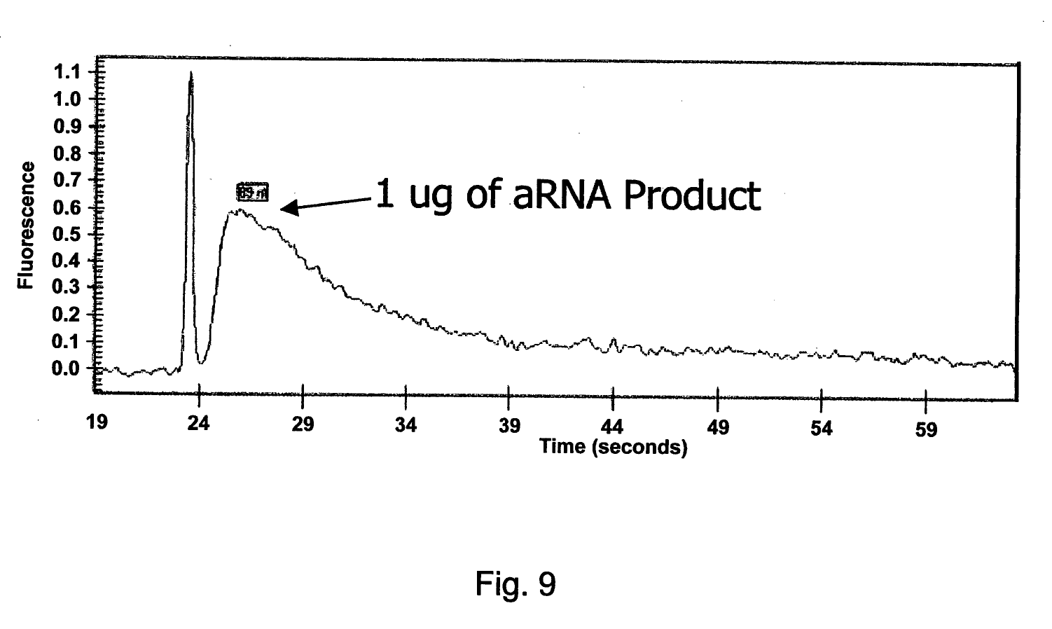

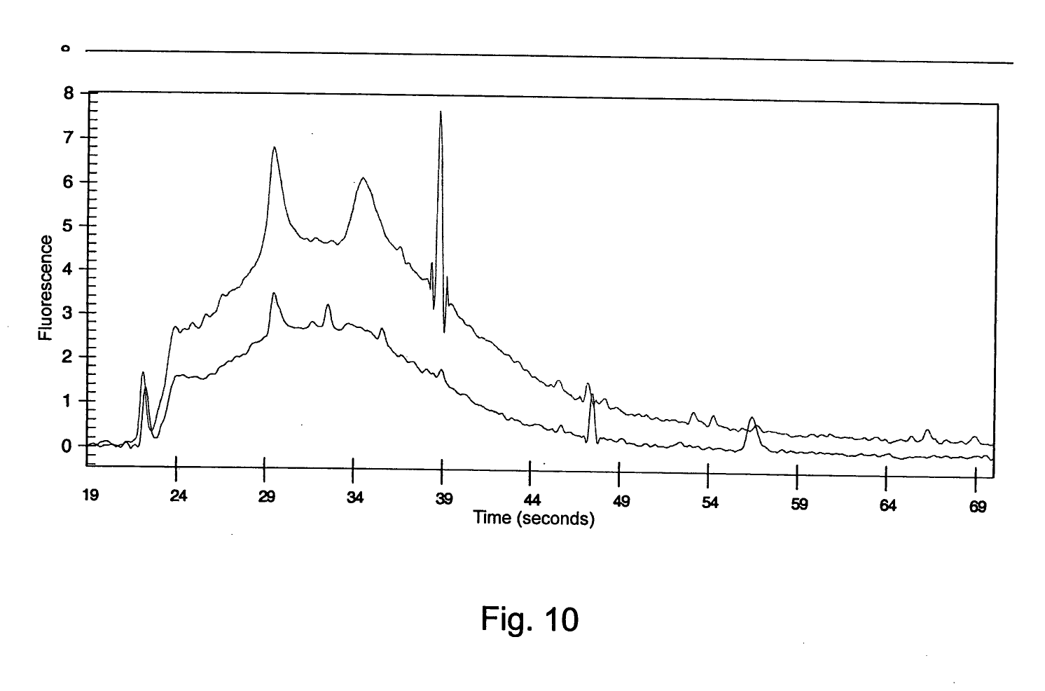

FIG. 9 illustrates results of a successful demonstration of an entire Van Gelder Eberwine synthesis on a microfluidic device as above. Sample RNA was captured on beads, and reverse transcriptase, second strand synthesis and in vitro transcription performed on the device. As illustrated in FIG. 9, 1 μg of an RNA product was produced. Additional experiments, the results of which are illustrated in FIG. 10 illustrates the results of amplifying RNA from total RNA. Two different time incubation periods were performed, with a peak corresponding to the RNA product being shown.

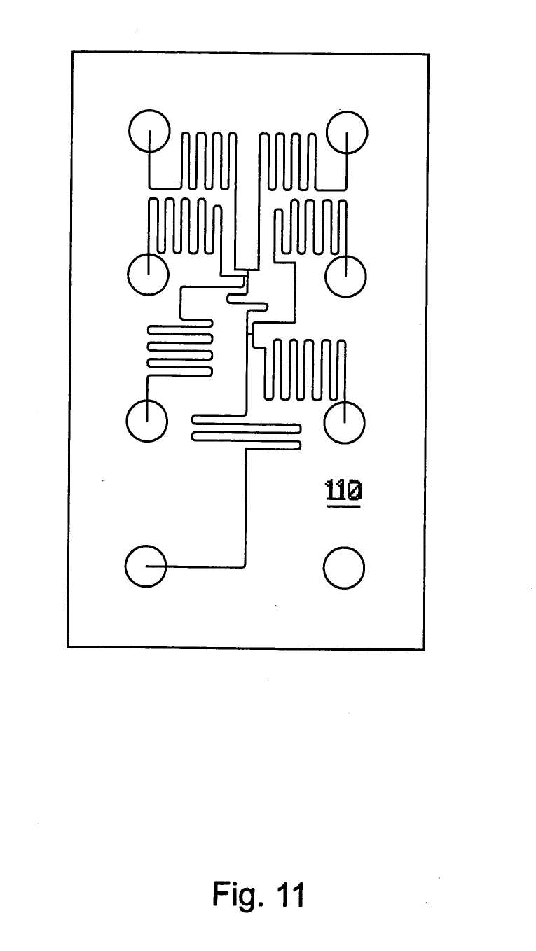

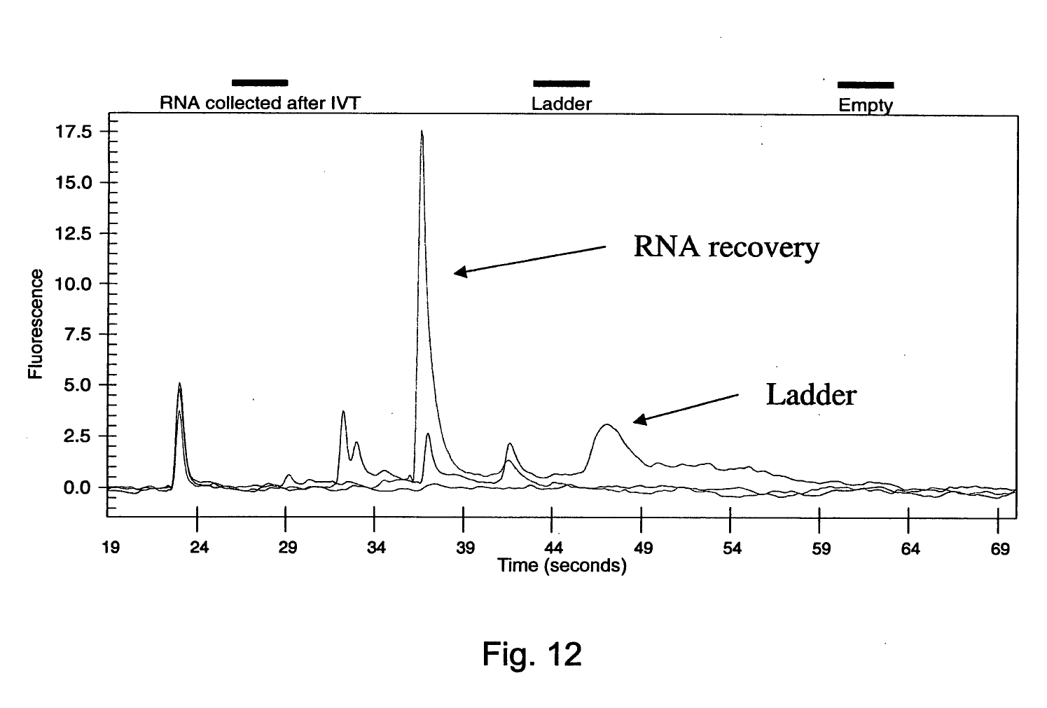

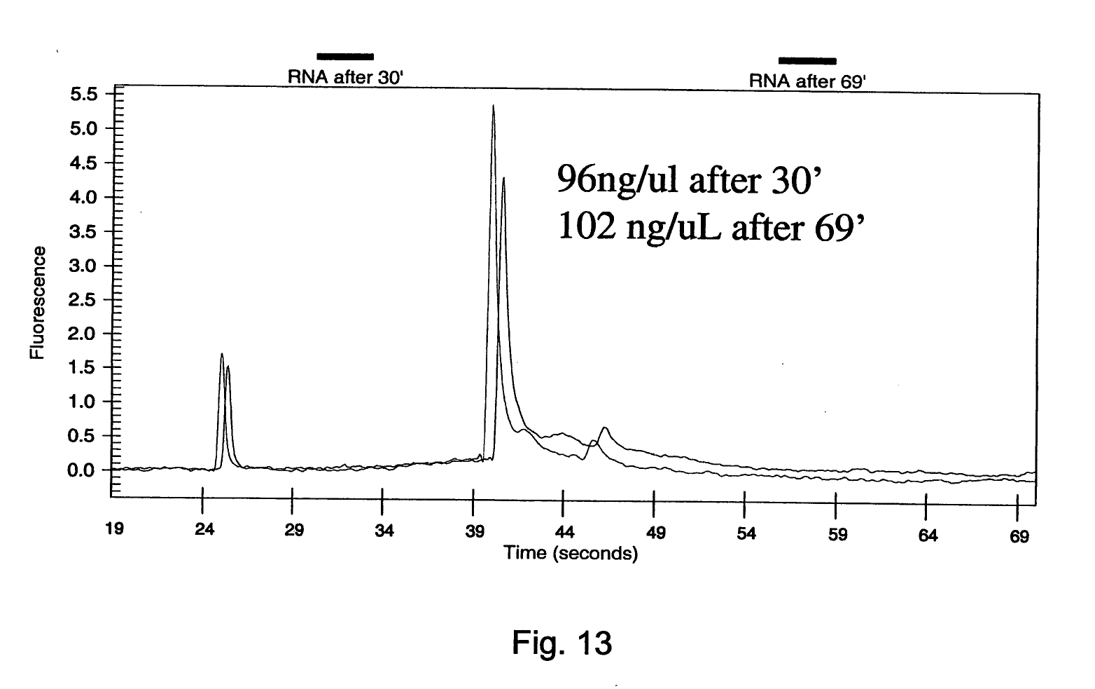

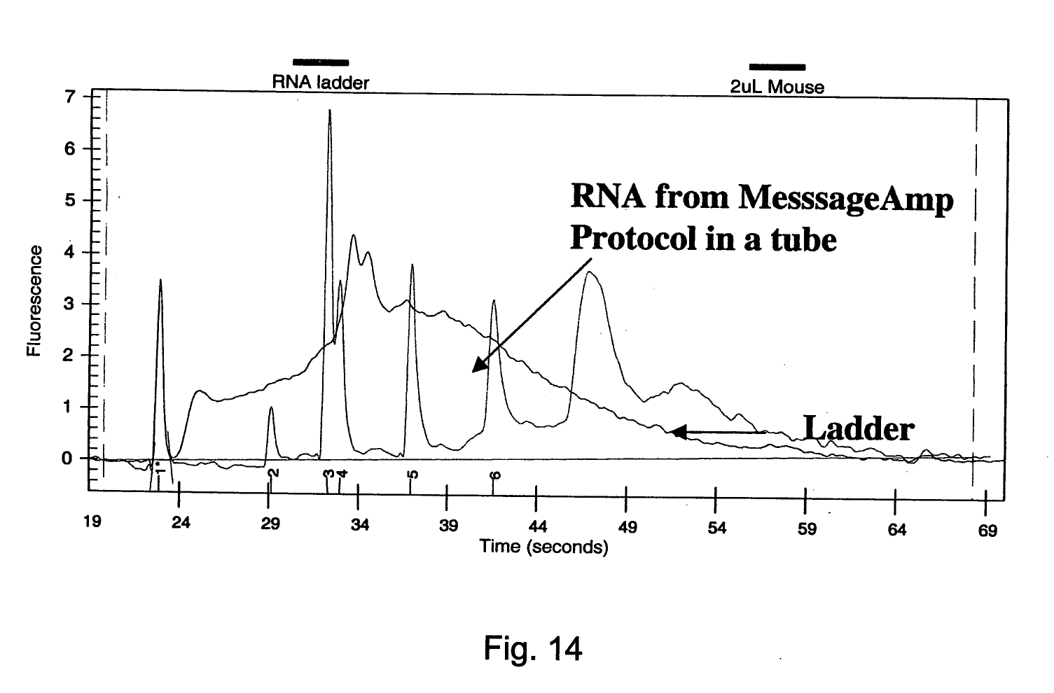

FIG. 11 illustrates an additional chip design. FIG. 12 illustrates results of an on device experiment performed with a device according to FIG. 11. The figure shows RNA produced on the chip, with 0.6 μL of 250 ng/μL amplified RNA being collected. This device included about 1000 beads in the bead bed and the reactions were performed at room temperature. In longer runs, the chip produced enough RNA product that it clogged. FIG. 13 shows results of an additional experiment, in which about 140 beads were loaded into the bead bed. Under a first set of reaction conditions, 1 μL of 96 ng/μL RNA was collected in 30 seconds (there was a 1 psi pressure difference driving flow). Under a second set of conditions, a 10 psi pressure difference was used to drive flow, resulting in 1.6 μL of material being collected after 69 seconds (102 ng/L). FIG. 14 shows results of a typical reaction run according to standard protocols for RNA amplification using a MessageAmp™ kit (available from Ambion), run on mouse RNA.

While the foregoing invention has been described in some detail for purposes of clarity and understanding, it will be clear to one skilled in the art from a reading of this disclosure that various changes in form and detail can be made without departing from the true scope of the invention. For example, all the techniques and systems/devices/apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of amplifying at least one RNA, the method comprising:
flowing one or more RNA amplification reagent into a microscale chamber; and, amplifying one or more template nucleic acid to produce one or more RNA amplicon in the microscale reaction chamber under conditions of constant or semi-constant flow, wherein one or more reaction parameter is optimized to provide production of the RNA amplicons in the reaction chamber, or flow of the amplicons out of the chamber, or both.

2. The method of claim 1, wherein the one or more RNA amplification reagents comprise one or more of: a solid support, the template nucleic acid, a DNA template, a poly(dT) oligonucleotide with an RNA polymerase promoter sequence, a cell, a cell extract, a reverse transcriptase, an rNTP, a dNTP, Mg++, or a buffer.

3. The method of claim 1, wherein amplifying the nucleic acid comprises expressing a DNA that encodes the RNA amplicon, in vitro.

4. The method of claim 1, wherein amplifying the one or more nucleic acids comprises expressing a plurality of cDNAs that encode total polyA mRNA from a biological sample.

5. The method of claim 4, wherein the biological sample comprises fewer than about 10 cells.

6. The method of claim 4, wherein the biological sample comprises about 1 cell.

7. The method of claim 4, wherein the biological sample comprises more than about 100 cells.

8. The method of claim 4, wherein the biological sample comprises more than about 1,000 cells.

9. The method of claim 1, wherein the reaction parameter is selected from the group consisting of: a rate of flow in the chamber, a temperature in the chamber, a concentration of one or more of the RNA amplification reagents in the chamber, inhibiting or enhancing DNA transcription in the amplification chamber, a channel size leading into or out of the chamber, a size of the chamber, a bead diameter of a bead bound to one or more additional RNA amplification reagent, total porosity of a bead bed bound to one or more additional RNA amplification reagent, a percent of fluid that diffuses in and out of a bead bed bound to one or more additional RNA amplification reagent as the fluid flows through and along the bead bed, residence time of reaction substrates or products through a bead bed, distance traveled by reaction substrates or products through a bead bed, and a direction of flow of one or more reactants or products through a bead bed.

10. The method of claim 1, wherein one or more additional RNA amplification reagent is contained within a bead bed that fills a deep portion of a microscale channel, wherein the channel comprises a lateral step up in depth, wherein the one or more amplification reagents and amplicons flow along a side of the bead bed and diffuse laterally in and out of the bead bed.

11. The method of claim 1, further comprising detecting the RNA amplicon.

12. The method of claim 11, wherein detecting the RNA amplicon comprises flowing the RNA amplicon into contact with an oligonucleotide array.

13. The method of claim 12, wherein the RNA amplicon is flowed out of the reaction chamber into contact with the array under constant flow conditions.

14. The method of claim 13, wherein the array is in one or more microchamber.

15. The method of claim 11, wherein detecting the RNA amplicon comprises real time detection or quantification of RNA amplicon formation.

16. The method of claim 11, wherein detecting the RNA amplicons comprises flowing an aliquot of a labeled amplicon past a fluorescence detector and determining a yield of the amplifying step.

17. The method of claim 11, wherein detecting the RNA amplicon comprises electrophoresing the amplicon through a matrix and detecting at least one resulting size separated RNA amplicon.

18. The method of claim 11, wherein detection of the RNA amplicon is a diagnostic or prognostic indicator for one or more polymorphism, SNP, disease or condition.

19. The method of claim 1, further comprising translating the RNA amplicons into one or more translation products.

20. The method of claim 19, wherein the translation products are detected or quantified in real time.

21. method of claim 19, wherein the translation products are detected by binding an antibody to the product and detecting binding of the antibody to the translation product.

22. The method of claim 19, wherein an in vitro translation reagent is contacted to the RNA amplicons under conditions of continuous or semi-continuous flow.

23. The method of claim 1, wherein amplifying the template nucleic acid comprises performing a Van Gelder-Eberwine series of reactions that converts one or more starting RNA into DNA by reverse transcription, performs a second strand synthesis to produce double stranded DNA and transcribes the double stranded DNA to produce the RNA amplicons.

24. The method of claim 1, wherein the amplifying step is performed twice in series, with the RNA amplicon from a first amplification reaction being used as the template nucleic acid for a second amplifying step.

25. The method of claim 24, wherein the second amplifying step comprises a Van-Gelder Eberwine reaction.

26. The method of claim 1, further comprising cleaving the RNA amplicon.

27. A method of detecting presence or absence of one or more target RNA in a biological sample, the method comprising:
performing one of more reverse transcription reaction on sample RNA from the biological sample to produce one or more cDNA;
flowing transcription reagents into contact with the one or more cDNA; performing one or more expression reaction on the cDNA under conditions of continuous or semi-continuous flow; and,
detecting one or more RNA amplicon, or lack thereof, produced by the expression reaction, thereby detecting presence or absence of the target RNA.
